(19) United States
(12) Patent Application Publication
(10) Pub. No.: US 2004/0116370 A1
(43) Pub. Date: Jun. 17, 2004

(54) Retroductal salivary gland genetic vaccination

(75) Inventors: Sean Tucker, San Francisco, CA (US); Michael Bennett, El Sobrante, CA (US); Yen-Ju Chen, Alameda, CA (US); David Olson, Alameda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genteric, Inc., Alameda, CA

(21) Appl. No.: 10/649,106

(22) Filed: Aug. 26, 2003

Related U.S. Application Data
(60) Provisional application No. 60/407,375, filed on Aug. 30, 2002.
(60) Provisional application No. 60/453,999, filed on Mar. 11, 2003.

(51) Int. Cl. A61K 48/00; A61K 31/655

(52) U.S. Cl. 514/44; 424/93.2; 514/150

(57) ABSTRACT

The present invention provides compositions and methods for eliciting an immune response and compositions and methods for transfecting antigen presenting cells.

### CROSS-REFERENCES TO RELATED APPLICATIONS

This application claims the benefit of U.S. Provisional Application Nos. 60/407,375 filed Aug. 30, 2002, and 60/453,999, filed Mar. 11, 2003 and U.S. patent application Ser. No. 10/639935, filed Aug. 12, 2003 (Bennett et al., “Polyionic Organic Acid Formulations,” Attorney Docket No. 020714-000720), the disclosures of which are hereby incorporated by reference in their entirety for all purposes.

### STATEMENT AS TO RIGHTS TO INVENTIONS MADE UNDER FEDERALLY SPONSORED RESEARCH AND DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK

Not applicable.

### BACKGROUND OF THE INVENTION

Genetic immunization is a promising technology that may advance vaccine efficacy and safety (see, e.g., Robinson and Torres, Semin. Immunol. 9:271 (1997) and Robinson et al., Int. J. Mol. Med. 4:549 (1999)). There are several well-characterized advantages of vaccines that use DNA to encode antigen rather than whole protein or live-attenuated viral vaccines. A hallmark of DNA vaccination is induction of cytotoxic T lymphocyte (CTL) activity that is superior to protein vaccination. Cellular immunity is enhanced because the protein being encoded by the DNA vector is processed and presented in a way that is analogous to the processing of viral antigens (see, e.g., Corr et al., J. Exp. Med. 184:1555 (1996)). In addition, plasmid-based vectors are much simpler to manufacture than protein-based or whole, live organism vaccines, and are considered safer to use. Finally, it is expected that it will be easier to incorporate new or altered antigens in DNA vaccines.

Protective immunity following DNA vaccination has been demonstrated in a variety of mouse models (see, e.g., Manickan et al., J. Immunol. 155:259 (1995) and Fynan et al., Proc. Natl. Acad. Sci. USA 90:11478 (1993)). However, reports from human studies have been less encouraging. The inability to scale with increasing body size is a problem common to delivery by injection into tissue. Part of the problem may be poor gene transfer resulting from limited distribution of DNA after injection into the muscle or skin, the predominant tissues used for DNA vaccination (see, e.g., Denis-Mize et al., Gene Ther. 7:2105 (2000)). Restricted DNA distribution results in a “needle-tract” effect. Although effective in small animals such as mice, gene transfer along a needle tract reaches only a small fraction of the total tissue in large animals and humans. Potential solutions that have been proposed include using electroporation to enhance transfection, or targeting DNA uptake to antigen presenting cells (APCs) (see, e.g., Singh et al., Proc. Natl. Acad. Sci. USA 92:811 (2000) and Mathiesen Gene Ther. 6:508 (1999). Another challenge for DNA vaccination is that the common routes of gene delivery typically provide suboptimal protection from pathogens invading through mucosal surfaces (see, e.g., Fynan et al., 1993, supra and Schreckenberger et al., Vaccine 19:227 (2000)). Although inoculation with a strong antigen by these routes may provoke a robust systemic CTL response, the mucosal immune response tends to be poor. The same is true with conventional vaccines (proteins or inactivated pathogens), where mucosal responses tend to be weak after intramuscular (i.m.) or other parenteral delivery (see, e.g., Mestecky et al. J. Clin. Immunol. 7:265 (1987) and Lue et al., Adv. Exp. Med. Biol. 371A:103 (1995)).

Broad mucosal protection has been difficult to achieve even when the antigens are delivered to mucosal tissues. This may be due in part to compartmentalization of immune activity. Mucosal immune responses tend to be intense at the site where the antigen is delivered, and are sometimes not well distributed throughout all mucosal sites (see, e.g., Johansson et al., Infect. Immun. 69:7481 (2001) and Forrester et al., Vaccines for Enteric Diseases T. Vesikari, ed., Tampere, Finland (2001)). For example, intranasal immunization tends to produce active mucosal responses in the lungs, some responses in the vagina, but more limited responses in the colon (see, e.g., Forrester et al., 2001, supra and Kuklin et al., J. Virol. 71:3138 (1997)).

Thus, there is a need in the art for new methods and compositions for genetic immunization. In particular, there is a need for methods of genetic immunization that induce a broad mucosal immune response.

### SUMMARY OF THE INVENTION

The present invention provides compositions and methods for genetic immunization, whereby a broad immune response is generated. In addition, the present invention provides compositions and methods for transfecting antigen presenting cells. In some embodiments antigen presenting cells associated with the mucosal immune system are transfected.

In one embodiment, the present invention provides a method for eliciting an immune response. An immunogenically effective amount of a composition comprising a nucleic acid encoding an immunogenic polypeptide is retroductally introduced, whereby an immune response is generated. In some embodiments the step of introducing is by cannulation. In other embodiments, the composition further comprises an adjuvant, such as, for example, cholera toxin or Al(OH)₃. In some embodiments, the composition is administered multiple times. In some embodiments, the nucleic acid is operably linked to an expression control sequence. In some embodiments, the nucleic acid is in a viral vector. In some embodiments, the immunogenic polypeptide is a cancer antigen. In other embodiments, the immunogenic polypeptide is a viral antigen such as, for example, HIV envelope protein or a portions thereof (e.g., gp160 or a portion thereof, gp 120 or a portion thereof, or gp41 or a portion thereof). In even other embodiments, the immunogenic polypeptide is a bacterial antigen, such as, for example anthrax protective antigen. In some embodiments, the composition further comprises a lipid (e.g., N,N,N′,N′-tetramethyl-N,N′-bis(2-hydroxyethyl)-2-3-di(oleoyloxy)-1,4-butanediammonium iodide.), whereby the lipid facilitates uptake of the nucleic acid by antigen presenting cells. In some embodiments, the salivary gland is a submandibular salivary gland, a parotid salivary gland, or a sublingual salivary gland. In other embodiments, the subject is a mammal, such as, for example, a primate, such as, for example, a human. In some embodiments, the immune response comprises a mucosal immune response.

In another embodiment, the present invention provides a method for transfecting antigen presenting cells. An immunogenically effective amount of a composition comprising a nucleic acid encoding an immunogenic polypeptide retroductally introduced into the lumen of a salivary gland duct of a subject. In some embodiments the step of introducing is by cannulation. In some embodiments, the composition is administered multiple times. In some embodiments, the nucleic acid is operably linked to an expression control sequence. In some embodiments, the nucleic acid is in a viral vector. In some embodiments, the immunogenic polypeptide is a cancer antigen. In other embodiments, the immunogenic polypeptide is a viral antigen such as, for example, HIV envelope protein or a portions thereof (e.g., gp 160 or a portion thereof, gp120 or a portion thereof, or gp41 or a portion thereof). In even other embodiments, the immunogenic polypeptide is a bacterial antigen, such as, for example anthrax protective antigen. In some embodiments, the composition further comprises a lipid, whereby the lipid facilitates uptake of the nucleic acid by the antigen presenting cells. In some embodiments, the salivary gland is a submandibular salivary gland, a parotid salivary gland, or a sublingual salivary gland. In other embodiments, the subject is a mammal, such as, for example, a primate, such as, for example, a human. In some embodiments, antigen presenting cells (e.g., dendritic cells) in a proximal lymph node are transformed by the nucleic acid. In other embodiments, the proximal lymph node is a draining lymph node. In even other embodiments, the draining lymph node is a cervical lymph node or a submandibular lymph node.

In a further embodiment, the present invention provides a pharmaceutical composition comprising a nucleic acid encoding an immunogenic polypeptide, a lipid, and a transition metal enhancer. The pharmaceutical composition elicits an immune response. In some embodiments, the lipid is N,N,N′,N′-tetramethyl-N,N′-bis(2-hydroxyethyl)-2-3-di(oleoyloxy)-1,4-butanediammonium iodide and the transition metal enhancer is ZnCl₂.

An “immunogenic composition” is one that elicits or modulates an immune response, preferably the composition induces or enhances an immune response in response to a particular antigen. Immune responses include humoral immune responses and cell-mediated immune responses. An immunogenic composition can be used therapeutically or prophylactically to treat or prevent disease at any stage.

A “salivary gland” is a gland of the oral cavity which secretes saliva, including the glandulae salivariae majores of the oral cavity (the parotid, sublingual, and submandibular glands) and the glandulae salivariae minores of the tongue, lips, cheeks, and palate (labial, buccal, molar, palatine, lingual, and anterior lingual glands).

“Retroductally introducing” refers to introduction of a composition through a duct in a salivary gland, wherein the composition flows through the salivary gland duct in a retrograde manner. Suitable ducts include all major and minor salivary gland ducts. For example the Wharton's duct or the Stenson's duct are suitable.

An “adjuvant” is a non-specific immune response enhancer. Suitable adjuvants include, for example, cholera toxin, Al(OH)₃, and polyionic organic acids. A “polyionic organic acid” (POD) as used herein, is typically a polyprotic polyaromatic organic compound wherein the compound contains at least two aromatic components. “Polyionic” compounds refer to compounds comprising one or more ionizable units, either as in the protonated form or as the conjugate salt. In certain embodiments, the PODS has associated therewith, such as complexed with, a transition metal enhancer as described below. One example of a polyionic organic acid is a dye. As used herein, a dye is a compound that absorbs radiation in the ultraviolet, visible and/or infrared regions of the electromagnetic spectrum. These regions of the electromagnetic spectrum correspond to radiation having wavelengths of 10⁻⁹ to 4×10⁻⁷, 4−7×10⁻⁷ and 7×10⁻⁷ to 10⁻⁴ meters, respectively. Dyes which are useful in the present invention include, but are not limited to, an acid dye, a disperse dye, a direct dye and a reactive dye. In a preferred embodiment, an acid dye is used. Suitable acid dyes include, but are not limited to, direct red dye, direct blue dye, acid black dye, an acid blue dye, an acid orange dye, an acid red dye, an acid violet dye, and an acid yellow dye. In certain other preferred embodiments, suitable acid dyes include, but are not limited to, Evans Blue, Congo Red, Ponceau S, Congo Corinth, Sirius red F3B, Ponceau 6R, amido black 10B, biebrich scarlet and aurintricarboxylic acid. In yet another preferred embodiment, a direct dye is used. Preferred direct dyes include direct red, direct blue, direct yellow and direct green. More preferably, direct blue 15 (Light Blue), direct red 28 (Congo Red) and direct blue 53 (Evans Blue) are used. Preferably, the dye absorbs in the visible light spectrum, between about 400 nm to 700 nm.

A “cationic lipid” refers to any of a number of lipid species which carry a net positive charge at a selective pH, such as physiological pH.

A “charge neutral lipid” or a “neutral lipid” refers to any of a number of lipid species which carry a net neutral charge at a selective pH, such as physiological pH.

An “anionic lipid” refers to any of a number of lipid species which carry a net negative charge at a selective pH, such as physiological pH.

“Mucosal immune responses” as used herein refers to immune responses generated in the mucosas of the gastrointestinal system (e.g., intestine, jejunum, ileum, duodenum,), the respiratory system; (e.g., lungs, trachea), and the urogenital tract (e.g., vagina, urethra) (see, e.g., Bannister et al. ed. (1995) Gray's Anatomy). Components of the mucosal immune system include, for example, tonsils, adenoids, Peyer's patches, appendix, and single lypmphoid follicles. Typically, there is a higher proportion of IgA produced in the mucosal immune response than in the peripheral immune response. A mucosal immune response includes both humoral aspects and cell mediated aspects.

“Humoral immune responses” or Th2-type responses” are mediated by cell free components of the blood, i.e., plasma or serum; transfer of the serum or plasma from one individual to another transfers immunity. Humoral immune responses include, for example, production of antigen-specific antibodies (e.g., neutralizing antibodies).

“Cell mediated immune responses” or “Th1-type responses” are mediated by antigen specific lymphocytes; transfer of the antigen specific lymphocytes from one individual to another transfers immunity. Cell mediated immune responses include, for example, development of antigen specific cytotoxicity, i.e., stimulation or activation of antigen-specific cytotoxic T cells.

“Antigen presenting cells” (APCs), as used herein refers to cells that are able to present immunogenic peptides or fragments thereof to T cell to activate or enhance an immune response. APCs include, for example, dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may be from any of a variety of biological fluids and organs, including tumor and peritumoral tissues, and may be autologous, allogeneic, syngeneic or xenogeneic cells.

“Lymph nodes” as used herein refers to any of the masses of lymphoid tissue which filter the flow of lymph (i.e., a body fluid that comprises lymphocytes). Lymph nodes are typically surrounded by a capsule of connective tissue, are distributed along the lymphatic vessels, and contain numerous lymphocytes, including antigen presenting cells. Lymph nodes include, for example, submandibular nodes, parotid nodes (i.e., superficial), buccal nodes, occipital nodes, cervical nodes (i.e., upper deep, lower deep, anterior, and superficial) submental nodes, infrahyoid nodes, retro-aurical nodes, jugulo-omohyoid nodes, jugulodigastric nodes, prelaryngeal nodes, pretracheal nodes, inguinal nodes, and intestinal mesentery nodes. A “draining lymph node” is a lymph node to which antigens or antigenic fragments are filtered by the lymph.

The immunogenic compositions (i.e., pharmaceutical compositions) of the present invention are administered to a subject in an amount sufficient to elicit an immune response in the subject. An amount adequate to accomplish this is defined as “immunogenically effective dose or amount.”

The term “protein” is used herein interchangeably with “polypeptide” and “peptide.”

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence. DNA regions are “operably linked” when they are functionally related to each other. For example, DNA for a signal peptide (secretory leader) is operably linked to DNA for a polypeptide if it is expressed as a precursor which participates in the secretion of the polypeptide; a promoter is “operably linked” to a coding sequence if it controls the transcription of the sequence; or a ribosome binding site is “operably linked” to a coding sequence if it is positioned so as to permit translation. Generally, “operably linked” means contiguous and, in the case of secretory leaders, in reading frame. DNA sequences encoding immunogenic polypeptides which are to be expressed in a microorganism will preferably contain no introns that could prematurely terminate transcription of DNA into mRNA.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

“Antibody” refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see, e.g. Fundamental Immunology (Paul ed., 4th ed. 1999). Thus, the term antibody, as used herein, also includes antibody fragments either produced by the modification of whole antibodies.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, mRNA, oligonucleotide, and polynucleotide.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99% or higher, compared to a reference sequence using the programs described herein, preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. “Substantial identity” of amino acid sequences for these purposes normally means that a polypeptide comprises a sequence that has at least 40% sequence identity to the reference sequence. Preferred percent identity of polypeptides can be any integer from 40% to 100%. More preferred embodiments include at least 60%, 65%, 70%, 75%, 80%, 85%, 90%, 95%, or 99%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Optimal alignment of sequences for comparison may be conducted by the local identity algorithm of Smith and Waterman (1981) Add. APL. Math. 2:482, by the identity alignment algorithm of Needleman and Wunsch (1970) J. Mol. Biol. 48:443, by the search for similarity method of Pearson and Lipman (1988) Proc. Natl. Acad. Sci. U.S.A. 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

A preferred example of algorithms that are suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

Another indication that nucleotide sequences are substantially identical is if two molecules hybridize to each other, or to a third nucleic acid, under moderately, and preferably highly, stringent conditions. Stringent conditions are sequence dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thennal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

For the purpose of the invention, suitable “moderately stringent conditions” include, for example, prewashing in a solution of 5×SSC, 0.5% SDS, 1.0 mM EDTA (pH 8.0), hybridizing at 50° C.-65° C., 5×SSC overnight, followed by washing twice at 65° C. for 20 minutes with each of 2×, 0.5× and 0.2×SSC (containing 0.1% SDS). Such hybridizing DNA sequences are also within the scope of this invention.

“Transition metal enhancer” as used herein refers to compounds having one or more transition metal atoms selected from the elements in Groups IIIB, IVB, VB, VIIB, VIIIB, IB, and IIB of the periodic table (i.e., the d-block) (see, e.g., Huheey, INORGANIC CHEMISTRY, Harper & Row, New York, 1983). The transition metals of the present invention also include those lanthanides (i.e., the first row of the f-block of the periodic table) and main group metals (i.e., groups IIIA, IVA, VA, and VIIA of the periodic table), having chemical properties similar to transition metal complexes.

### BRIEF DESCRIPTION OF THE DRAWINGS

FIG. 1 illustrates expression of hGH within the submandimular gland with retreatment. The right and left submandibular glands of rats were each treated with 200 μL of water containing 175 μg of hGH plasmid DNA on day 0. Retreated animals received a second DNA dose on Day 21. The animals were sacrificed and their submandibular glands were harvested on either Day 7 or Day 28. N=8 for each group. Error bars=SEM.

FIG. 2 illustrates antibody responses following salivary gland DNA administration. FIG. 2A illustrates measurement of IgG and IgA antibody isotypes 3 weeks after 350 μg of plasmid DNA encoding hGH was infused retroductally into the salivary glands (submandibular) or injected intramuscular. As a control, 15 μg of hGH protein was injected subcutaneously (s.c.) in a 50/50 mixture of sterile saline/ Freund's complete adjuvant. N=6 for DNA groups, N=4 animals for the s.c. group, and N=3 for the untreated group. FIG. 2B illustrates IgG plasma titers over time following DNA administration. In this experiment, submucosal (submucol) refers to needle injection into the tissue below the tongue. Salivary gland (SG), intramuscular (i.m.), and the submucol groups were given one dose of 175 μg DNA at the time shown by the arrow. N=6 for DNA treated groups, N=3 for untreated.

FIG. 3 illustrates antibody responses to gp120 following salivary gland and intramuscular DNA administration. Antibody responses were measured after plasmid DNA was retroductally infused into the salivary gland (SG) or injected intramuscular (i.m.). FIG. 3A. illustrates measurement of anti-HIV gp120 in the plasma of rats treated by two doses of 175 μg DNA given four weeks apart. The plasma titers were measured 2 weeks after the last dose. These experiments were performed with either DNA and water (SG) or DNA and sterile saline (i.m.). hGH DNA administered to the SMG (hGH/SG) represents an irrelevant DNA control in this experiment, and controls for any non-specific immune responses resulting due to DNA delivery to the SG. N=6 animals per group except for protein and untreated groups (N=3). FIG. 3B illustrates measurement of anti-gp120 over time. In this experiment, Zn²⁺ was added to enhance expression (20, 40). N=6 animals in DNA vaccinated group and N=3 in the untreated group. Error bars represent the standard error of the mean (SEM). Arrows represent the times the treated animals were given 175 μg DNA.

FIG. 4 illustrates anti-gp120 T cell responses to antigen. FIG. 4A illustrates T cell activity specific to gp120 measured by γ-IFN ELISA of the cell supemate. The stimulation index represents the ratio between the γ-IFN secreted by gp120 stimulated cells divided by the γ-IFN secreted by cultured but not antigen stimulated cells. Two concentrations of antigen were provided to the cultured splenocytes, 0.2 μg gp120 (solid) and 1.0 μg gp120 (open). DNA vaccinated groups were treated 3 times with 175 μg gp120 DNA per treatment, with the last DNA dose given 1 week before spleen harvest. FIG. 4B illustrates CD4 and CD8 T cell responses measured by intracellular γ-IFN. The percentage of γ-IFN+ cells from each T cell subset was determined by flow cytometry. DNA vaccinated animals were treated with 175 μg gp120 (or 175 μg hGH) on weeks 0, 4, and 8 weeks and harvested on week 9. N=6 for gp120 /SG, gp120/i.m; N=4 for hGH/SG and protein; and N=3 for untreated. Error bars=SEM.

FIG. 5 illustrates mucosal immune responses within saliva following salivary gland DNA vaccination. FIG. 5A illustrates anti-hGH IgA was measured by ELISA. Four vaccinated animals (1-4) were compared to two untreated animals (5-6) at 3 weeks after a single dose given to the SG. Each saliva sample was diluted to a concentration of 12.5 ng/ml total IgA prior to the anti-hGH IgA ELISA. One saliva sample, shown with an * was diluted to 6.25 ng/ml total IgA due to insufficient material. The O.D. values were then plotted for individual animals. FIG. 5B illustrates anti-hGH secretory component measured by ELISA. Twenty five saliva samples from the same five animals were used as starting material to measure titer. Results are presented as hGH specific titers.

FIGS. 6A-C illustrate immune responses following salivary gland DNA vaccination. (A) Anti-gp 120 IgA and IgG were measured by ELISA. Salivary gland vaccinated animals were compared to untreated animals at 6 weeks post initial vaccination for their fecal IgA response and saliva IgA response to gp120. Saliva demonstrated statistically significant responses as compared to untreated (p=0.02 by Student's T test). In a separate experiment using female rats, specific IgG was compared after normalizing to use equivalent amounts of IgG. Salivary gland treated animals produced significant vaginal IgG responses (N=4) compared to the untreated animals (p=0.02). (B) Cells were isolated from Peyer's patches 1 week after the last DNA administration from a variety of vaccine groups. The isolated cells were placed in ELISPOT plates coated with gp120 protein to measure the numbers of ASC that recognize the antigen. All DNA groups (N=6) were given 175 μg gp120 DNA on 0, 4, and 8 weeks and harvested on week 9. N=3 for protein and untreated groups. Error bars represent the SEM. SG was better than i.m. for producing Peyer's patch ASC by Student's T test (p=0.03) (C) Lung lavages were analyzed for specific antibodies to gp120 21 weeks after the last salivary gland vaccination (SG). Samples were normalized to 25 ng/ml IgA (for measurement of specific IgA and secretory component (s.c.)) and 100 ng/ml total IgG before determining an OD value by ELISA. IgA and secretory specific responses were found to be statistically significant (p=0.046, p=0.02, respectively).

FIG. 7 illustrates plasma antibody titers in dogs following parotid gland retroductal DNA delivery. On day 0, 2.5 mg of plasmid DNA encoding hGH or 2.5 mg of plasmid DNA encoding secreted alkaline phosphatase (SEAP) was retroductally delivered to the parotid salivary glands of 10 kg dogs in a total volume of 700 μl with 2 mg/ml Evans Blue. On day 7, 0, 5.25 mg of plasmid DNA encoding hGH or 5.25 mg of plasmid DNA encoding secreted alkaline phosphatase (SEAP) was retroductally delivered to the parotid salivary glands of 10 kg dogs in a total volume of 3000 μl with 2 mg/ml Evans Blue. Anti-hGH IgG was measured 2, 19, and 33 days after the second infusion of DNA. N=2 for the hGH DNA group, N=2 for the unrelated antigen group. Antibody titers to hGH protein were greater than 5,000. Results are presented as hGH specific titers.

FIG. 8 illustrates enhancement of genetic immunization by co-formulation of the nucleic acid with lipid. DNA encoding hGH (human growth hormone) was co-formulated with ZnCl₂ alone or with ZnCl₂ and 200 μg DOHBD:DOPE (3:1). 88 μg DNA encoding hGH was administered per submandibular salivary gland on weeks 0 and 6. Anti-hGH IgA was measured on week 9 after normalizing the amount of total IgA in each sample. The response at two different concentrations of total IgA is shown. Results show that 3 out of 3 rats had high responds in the Lipid/Zn group and 2 out of 6 responded in the no lipid/Zn group.

FIG. 9 illustrates anti-PA (anthrax protective antigen) response following retroductal DNA delivery to the submandibular gland of Sprague/Dawley rats. Animals were treated on week 0 with 175 μg DNA per gland in 200 μl water with 4 mg/ml Congo Red. The antibody titers were examined 3 weeks later for plasma antibody responses. N=6 for DNA vaccinated, N=3 for untreated.

FIG. 10 illustrates distal mucosal immune response following genetic immunization. 100 μg DNA encoding hGH (i.e., plasmid pFOXCMVhuGH-G3) in 100 μl distilled, deionized H₂0 was retroductally delivered to the submandibular salivary glands of Sprague Dawley rats on weeks 0 and 8. On week 12, lung lavages were collected. Anti-hGH IgA was detected using an ELISA.

FIG. 11 illustrates HIV neutralization following genetic immunization. 88 μg DNA encoding HIV envelope protein gp120 in 200 μl distilled, deionized H₂0 was retroductally delivered to the submandibular salivary glands of Sprague Dawley rats on weeks 0 and 3. The DNA was delivered alone or in a formulation comprising: Congo Red (6 mg/ml), Congo Red (6 mg/ml )/DOHBD:DOPE (3:1)/Zn (0.125 mM), or aurintricarboxylic acid/Zn (0.125 mM). On week 9, plasma samples were collected and HIV neturalization assays were performed.

FIG. 12 illustrates a comparison of anti-anthrax protective antigen (PA) plasma IgG titers from retroductal introduction of formulations with DNA encoding PA with or without a polyionic organic acid into the salivary gland of rats.

FIG. 13 illustrates a time course comparing anti-anthrax protective antigen (PA) plasma IgG titers using different introduction methods and positive (PA protein) and negative (hGH DNA ) controls. Antibody titers were measured following retroductal delivery of PA DNA to the salivary gland (SG/PA DNA), injection of PA DNA into the muscle (i.m./PA DNA), or retroductal delivery of hGH DNA to the salivary gland (SG/hGH DNA). Subcutaneous PA protein plus CFA vaccination (s.c./Prtn+CFA), and naive animals served as positive and negative controls respectively. Arrows indicate when DNA or protein was administered.

FIGS. 14A-B illustrate hGH expression in tissue and anti-hGH responses in the plasma of rats following salivary gland retroductal delivery. (A) DNA was formulated with either a ZnLipid combination (0.125 mM Zn, 3:1 DOHBD:DOPE Lipid) or a 3.6 mM Zn formulation and administered at 0 and 6 weeks to rat submandibular glands (SMG). The antibody responses were measured at 8 wks post initial DNA administration for either IgG or IgA. (B) DNA was formulated in Lipid (3:1 DOHBD:DOPE) with increasing Zn concentrations and infused into the SMG of rats. 48 hours after delivery, glands were harvested and analyzed for hGH expression. N=8 for each group.

FIGS. 15A-B illustrate mucosal immune responses in saliva and lungs following salivary gland vaccination. Salivary gland vaccination was compared using different DNA formulations. Either Znlipid, or Zn was co-formulated with plasmid DNA before vaccinating at weeks 0 and 3. (A) Stimulated saliva samples were normalized to use equivalent amounts of total IgA before reading the specific saliva ELISA O.D. values. Results from both 25 and 3.1 ng/ml total IgA are shown at week 9 for anti-hGH specific IgA. (B) Specific Lung IgA responses to gp120 on week 14 using the ZnLipid formulation.

FIGS. 16A-B illustrate mucosal immune responses in plasma, saliva, and fecal samples following salivary gland vaccination using different adjuvants. Either CR, ZnLipid, CTb, or water (ddH2O) were co-formulated with DNA encoding gp 120 before vaccinating on weeks 0, 3. (A) Saliva samples were normalized to use equivalent amounts of total IgA before measuring the specific saliva response at weeks 6 and 9 by ELISA. (B) Fecal samples were measured on week 9 by ELISA.

FIG. 17 illustrates antibody (IgA) responses in saliva samples following salivary gland vaccination. Rat salivary glands were vaccinated by retroductal DNA administration on weeks 0 and 3. Plasmid DNA was co-formulated with either LipidZn, CR, or EB. Stimulated saliva samples were normalized to use equivalent amounts of total IgA before measuring the specific IgA response to HIV gp120. Data are plotted for the 6 week time point.

### DETAILED DESCRIPTION OF THE INVENTION

### I. Introduction

The present invention provides methods and compositions for eliciting immune responses and for transfecting antigen presenting cells by retroductal delivery of compositions comprising nucleic acids encoding an immunogenic polypeptide to the lumen of a salivary gland duct. The invention is based on the surprising discovery that retroductal delivery of nucleic acids is particularly effective for eliciting immune responses specific for the immunogenic peptide encoded by the nucleic acid and for transfecting antigen presenting cells.

Salivary glands have been used as depot organs for gene transfer and therapeutic protein expression (see, e.g., Goldfine et al., Nat. Biotechnol. 15:1378 (1997)). Unlike the skin or muscle, the anatomy and physiology of the salivary glands makes them ideal candidates as platforms for gene delivery and enhanced protein expression (see, e.g., Goldfine et al., 1997, supra and Baum and O'Connell, Crit. Rev. Oral Biol. Med. 10:276 (1999)). These organs produce and secrete large amounts of protein. The secreted protein is detected both in the blood and the saliva (see, e.g., Hoque et al., Hum. Gene Ther. 12:1333 (2001) and Baum et al., Hum. Gene Ther. 10:2789 (1999)). The major glands (parotid and submandibular) can be accessed by non-surgical means through the duct that opens into the oral cavity. The ductal nature of these glands allows for simple and direct application of aqueous material, because retroductal infusion provides for a near complete exposure of the target cells to the gene vector without dilution. Importantly, retroductal delivery perfuses the entire organ regardless of the size of the animal, so delivery is scalable from mice to men.

Without intending to be bound by theory, it is proposed that retroductal administration of compositions comprising nucleic acids encoding immunogenic polypeptides as disclosed herein results in both direct and indirect priming of T cells. For direct priming of T cells, the retroductally introduced nucleic acids encoding immunogenic polypeptides directly transfect APC, e.g., dendritic cells, which then present the immunogenic peptides to T lymphocytes, thereby generating an immune response specific for the immunogenic peptide. For indirect priming of T cells, the retroductally introduced nucleic acids encoding immunogenic polypeptides transfect non-professional antigen presenting cells, which then express the immunogenic polypeptides. The expressed immunogenic polypeptides are picked up by APC, e.g., dendritic cells, which then present the immunogenic peptides to T lymphocytes, thereby generating an immune response specific for the immunogenic peptide. For both direct and indirect priming of T cells, the transfection may occur within the salivary gland or within a proximal lymph node.

If a professional APC expresses an antigen and is able to stimulate or activate a T cell, the stimulation or activation is referred to as direct priming of the T cell. If a non-professional antigen presenting cell (i.e., any cell expressing Class I MHC) expresses an antigen, and that antigen is picked up by an APC that in turn stimulates or activates a T cell, the stimulation or activation is referred to as indirect priming of the T cell since the cell that is stimulating the T cell is not the same cell that is expressing the antigen.

A composition comprising a nucleic acid encoding an immunogenic polypeptide is retroductally delivered to the lumen of a salivary gland duct such that the immunogenic polypeptide or a fragment thereof is presented on the surface of the antigen presenting cell. Any APC may be transfected, including, professional APC such as for example, dendritic cells, B cells or macrophages, and nonprofessional APC. Typically dendritic cells or progenitors thereof are transfected by the methods of the present invention. Dendritic cells are highly potent APCs (Banchereau et al. (1998) Nature 392:245-251). In general, dendritic cells may be identified based on their typical shape (stellate in situ, with marked cytoplasmic processes (dendrites) visible in vitro), their ability to take up, process and present antigens with high efficiency, and their ability to activate naive T cell responses.

Dendritic cells and their progenitors are found at low levels in peripheral blood, bone marrow, tumor-infiltrating cells, peritumoral tissues-infiltrating cells, lymph nodes, spleen, skin, umbilical cord blood, lamina propria and Peyer's patches. Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APCs with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB). Isolation of dendritic cells and their progenitors is a complicated, labor-intensive process and generally results in very few useful antigen presenting cells. Moreover, the isolated dendritic cells are difficult to transform. The present invention overcomes the problems associated with isolation and transfection of dendritic cells by providing a method to transfect antigen presenting cells (e.g., dendritic cells) in vivo by retroductally delivering compositions comprising nucleic acids encoding immunogenic polypeptides to the lumen of the salivary gland duct. The methods and compositions of the present invention are particularly useful for transfecting dendritic cells associated with the mucosal immune system.

### II. Compositions of the Present Invention

One embodiment of the present invention provides compositions (i.e., pharmaceutical compositions) comprising a nucleic acid encoding an immunogenic polypeptide. As described in detail below, the compositions may further comprise a lipid and/or a non-lipid compound, and/or a transition metal enhancer.

A. Immunogenic Polypeptides

Nucleic acids encoding suitable immunogenic polypeptides may be derived from antigens, such as, for example, cancer antigens, bacterial antigens, viral antigens, fungal antigens, or parasite antigens. Cancer antigens include, for example, antigens expressed, for example, in colon cancer, stomach cancer, liver cancer, pancreatic cancer, lung cancer, ovarian cancer, prostate cancer, breast cancer, skin cancer (e.g., melanoma), leukemia, lymphoma, or myeloma. Exemplary cancer antigens include, for example, HPV L1, HPV L2, HPV E1, HPV E2, PSA, placental alkaline phosphatase, AFP, BRCA1, Her2/neu, CA 15-3, CA 19-9, CA-125, CEA, hCG, urokinase-type plasminogen activator (uPA), plasminogen activator inhibitor and MAGE-1. Bacterial antigens may be derived from, for example, Staphylococcus aureus, Staphylococcus epidermis, Helicobacter pylori, Streptococcus bovis, Streptococcus pyogenes, Streptococcus pneumoniae, Listeria monocytogenes, Mycobacterium tuberculosis, Mycobacterium leprae, Corynebacterium diphtheriae, Borrelia burgdorferi, Bacillus anthracis, Bacillus cereus, Clostridium botulinum, Clostridium difficile, Salmonella typhi, Vibrio chloerae, Haemophilus influenzae, Bordetella pertussis, Yersinia pestis, Neisseria gonorrhoeae, Treponema pallidum, Mycoplasm sp., Neisseria meningitidis, Legionella pneumophila, Rickettsia typhi, Chlamydia trachomatis, and Shigella dysenteriae. Viral antigens may be derived from, for example, human immunodeficiency virus, human papilloma virus, Epstein Barr virus, herpes simplex virus, human herpes virus, rhinoviruses, cocksackieviruses, enteroviruses, hepatitis A, hepatitis B, hepatitis C, and hepatitis E, rotaviruses, mumps virus, rubella virus, measles virus, poliovirus, smallpox virus, influenza virus, rabies virus, and Variella-zoster virus. Fungal antigens may be derived from, for example, Tinea pedis, Tinea corporus, Tinea cruris, Tinea unguium, Cladosporium carionii, Coccidioides immitis, Candida sp., Aspergillus fumigatus, and Pneumocystis carinii. Parasite antigens may be derived from, for example, Giardia lamblia, Leishmania sp., Trypanosoma sp., Trichomonas sp., Plasmodium sp., and Schistosoma sp.

The nucleic acids encoding immunogenic polypeptides, are typically produced by recombinant DNA methods (see, e.g., Ausubel, et al. ed. (2001) Current Protocols in Molecular Biology). For example, the DNA sequences encoding the immunogenic polypeptide can be assembled from cDNA fragments and short oligonucleotide linkers, or from a series of oligonucleotides, or amplified from cDNA using appropriate primers to provide a synthetic gene which is capable of being inserted in a recombinant expression vector (i.e., a plasmid vector or a viral vector) and expressed in a recombinant transcriptional unit. Once the nucleic acid encoding an immunogenic polypeptide is produced, it may be inserted into a recombinant expression vector that is suitable for in vivo expression.

Recombinant expression vectors contain a DNA sequence encoding an immunogenic polypeptide operably linked to suitable transcriptional or translational regulatory elements derived from mammalian or viral genes. Such regulatory elements include a transcriptional promoter, an optional operator sequence to control transcription, a sequence encoding suitable mRNA ribosomal binding sites, and sequences which control the termination of transcription and translation. An origin of replication and a selectable marker to facilitate recognition of transformants may additionally be incorporated.

The transcriptional and translational control sequences in expression vectors to be used in transforming vertebrate cells in vivo may be provided by viral sources. For example, commonly used promoters and enhancers are derived, e.g., from adenovirus, simian virus (SV40), and human cytomegalovirus. For example, vectors allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in mammalian cells are suitable. Further viral genomic promoter, control and/or signal sequences may be used, provided such control sequences are compatible with the host cell chosen.

Suitable vectors include, for example, herpes simplex virus vectors as described in Lilley et al., Curr. Gene Ther. 1(4):339-58 (2001), alphavirus DNA and particle replicons as decribed in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in , e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769; Lebkowski et al. (1988) Mol. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875). Additional suitable vectors include E1B gene-attenuated replicating adenoviruses described in, e.g., Kim et al., Cancer Gene Ther.9(9):725-36 (2002) and nonreplicating adenovirus vectors described in e.g., Pascual et al., J. Immunol. 160(9):4465-72 (1998) Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an immunogenic polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

Other known viral-based delivery systems are described in, e.g., Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; Guzman et al. (1993) Cir. Res. 73:1202-1207; and Lotze and Kost, Cancer Gene Ther. 9(8):692-9 (2002).

B. Transition Metal Enhancers

In some embodiments of the present invention, the composition further comprises a transition metal enhancer. Suitable transition metal enhancers include, for example, zinc chloride, zinc acetate, zinc bromide, zinc carbonate, zinc citrate, zinc fluoride, zinc halide, zinc hydroxide, zinc iodide, zinc nitrate, zinc oxide, zinc selenide, zinc sulfate, zinc telluride, or mixtures thereof. Other suitable transition metal enhancers include, for example, CuCl₂, CoCl₂, NiCl₂, and MgSO₄ (Shiokawa et al., Biochem J. 326:675 (1997) and Torriglia et al., Biochimie 79:435 (1997)). Other suitable transition metal enhancers are described in U.S. Pat. No. 6,372,722, U.S. patent application Ser. No. 09/766,320, filed Jan. 18, 2001, and WO 01/52903, filed Jan. 19, 2001.

Transition metals enhancers that are useful include copper containing compounds such as, for example. In some embodiments, the transition metal enhancer is a nickel, cobalt, copper, aluminum or gallium halide. In some embodiments, the transition metal enhancer is NiCl₂, CoCl₂, CuCl₂, AICl₂, or GaCI₂.

In other embodiments, the transition metal enhancers is a zinc ammonium complex together with its counter ion, zinc antimonide, zinc arsenate, zinc arsenide, zinc arsenite, zinc benzoate, zinc borate (Zn₂Z₆O₁₁), zinc perborate, zinc bromide, zinc butyrate, zinc carbonate, zinc chromate, zinc chrome, zinc chromite, zinc citrate, zinc decanoate, zinc dichromate, zinc dimer, zinc ethylenebis(dithiocarbamate), zinc fluoride, zinc formate, zinc gluconate, zinc glycerate, zinc glycolate, zinc hydroxide, zinc iodide, zinc lactate, zinc methoxyethoxide, zinc naphthenate, zinc nitrate, zinc nitrate hexahydrate, zinc nitrate trihydrate, zinc octanoate, zinc oleate, zinc oxide, zinc pentanoate, zinc perchlorate hexahydrate, zinc peroxide, zinc phenolsulfonate, zinc propionate, zinc propylenebis(dithiocarbamate), zinc stannate, zinc stearate, zinc sulfate, zinc titanate, zinc tetrafluoroborate, and zinc trifluoromethanesulfonate.

Additional transition metal enhancers that may be used according to the methods of the present invention include, for example, cobaltous nitrate, cobaltous oxide, cobaltic oxide, cobalt nitrite, cobaltic phosphate, cobaltous chloride, cobaltic chloride, cobaltous carbonate, chromous acetate, chromic acetate, chromic bromide, chromous chloride, chromic fluoride, chromous oxide, chromium dioxide, chromic oxide, chromic sulfite, chromous sulfate heptahydrate, chromic sulfate, chromic formate, chromic hexanoate, chromium oxychloride, chromic phosphite, cuprous oxide, cupric oxide, cupric chloride, cuprous acetate, cuprous oxide, cuprous chloride, cupric acetate, cupric bromide, cupric chloride, cupric iodide, cupric oxide, cupric sulfate and cupric sulfide, cupric propionate, cupric acetate, cupric metaborate, cupric benzoate, cupric formate, cupric dodecanoate, cupric nitrite; cupric oxychloride, cupric palmitate, cupric salicylate, manganese iodide, mangnese sulfate, manganous acetate, manganous benzoate, manganous carbonate, manganese chloride, manganese bromide, manganese dichloride, manganese trichloride, manganous citrate, manganous formate, manganous nitrate, manganous oxalate, manganese monooxide, manganese dioxide, manganese trioxide, manganese heptoxide, manganic phosphate, manganous pyrophosphate, manganic metaphosphate, manganous hypophosphite, manganous valerate, ferrous acetate, ferric benzoate, ferrous bromide, ferrous carbonate, ferric formate, ferrous lactate, ferrous nitrate, ferrous oxide, ferric oxide, ferric acetate, ferric hypophosphite, ferric sulfate, ferrous sulfite, ferric hydrosulfite, ferrous bromide, ferric bromide, ferrous chloride, ferric chloride, ferrous iodide, ferric iodide, nickel acetylacetonate, nickel bromide, nickel carbonate, nickel chloride, nickel cyanide, nickel dibromide, nickel dichloride, nickel dioleate, nickel fluoride, nickel fluoroborate, nickel hydroxide, nickel methylate, nickel nitrate, nickel nitrate hexahydrate, nickel oxide, nickel stearate, nickel sulfate, nickel sulfite, nickel thallate, or nickel salts of other organic acids such as ricinoleic acid, cobalt chloride, cobalt fluoride, cobalt nitrate, cobalt sulfate, cobalt octoate, cobalt fluoroborate, cobalt stearate, cobalt oxide, cobalt hydroxide, cobaltous bromide, cobaltous chloride, cobalt butylate, cobaltous nitrate hexahydrate, or mixtures thereof.

In a some embodiments of the present invention, the transition metal enhancers of the present invention are free metals, complexes, adducts, clusters, and/or salts of zinc, copper, nickel, cobalt, aluminum or gallium.

C. Lipids and Non-Lipid Compounds

In some embodiments of the present invention, the compositions further comprise a lipid or a non-lipid compound that binds to the nucleic acid. Without intending to be bound by theory, it is proposed that the lipids or other compounds enhance transfection efficiency by serving as an adjuvant or by enhancing target cell absorption of the nucleic acid.

Typically the lipid is a cationic lipid. Suitable cationic lipids include, for example, N,N,N′,N′-tetramethyl-N,N′-bis(2-hydroxyethyl)-2-3-di(oleoyloxy)-1,4-butanediammonium iodide (DOHBD), N,N[bis (2-hydroxyethyl)-N-methyl-N-[2,3-di(tetradedecanoyloxy)propyl]ammonium chloride, N,N-dioleyl-N,N-dimethylammonium chloride (“DODAC”), N-(2,3-dioleyloxy)propyl)-N,N,N-trimethylammonium chloride (“DOTMA”), N,N-distearyl-N,N-dimethylammonium bromide (“DDAB”), N-(2,3-dioleoyloxy)propyl)-N,N,N-trimethylammonium chloride (“DOTAP”), 3-(N-(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”), (“E-DLPC”), (“E-DMPC”) and (“E-DPPC”). Other cationic lipids suitable for use in the present invention are disclosed in for example, U.S. Pat. Nos. 5,527,928, 5,744,625, 5,892,071, 5,869,715, 5,824,812, 5,925,623 and 6,043,390. Additional suitable lipids are described in U.S. patent application Ser. No. 09/766,320, filed Jan. 18, 2001, and WO 01/52903, filed Jan. 19, 2001. Poly(lactide-co-glycolide) (PLG)-based cationic microparticles as described in Singh et al., 2000, supra, can also be used for retroductal delivery of compositions comprising nucleic acids according to the methods of the present invention. The cationic lipid may be used alone, or complexed with a charge neutral lipid before co-formulation with the nucleic acid. Typically the cationic lipid is DOHBD. The cationic may be used alone or complexed with a charge neutral lipid such as, for example, dioleoylphosphatidylethanolamine (DOPE), palmitoyloleoylphosphatidylcholine (POPC), egg phosphatidylcholine (EPC), distearoylphosphatidylcholine (DSPC), cholesterol. Typically, the charge neutral lipid is DOPE. The cationic lipid:charge neutral lipid ratio in the complex may be about 1:1, 2:1, 3:1, 4:1, 5:1, 6:1, 7:1, 8:1, 9:1, 10:1, 1:2, 1:3, 1:4, 1:5, 1:6, 1:7, 1:8, 1:19, or 1:10. Typically, the cationic lipid:charge neutral lipid is 3:1.

Typically the amount of lipid present in the cationic lipid/DNA/transition metal is expressed in terms of the cationic lipid:DNA phosphate charge ratio. Suitable charge ratios include, for example, about 0.1, 0.25, 0.35, 0.4, 0.5, 0.75, 1.0, 1.5, and 2.0. Typically, the charge ratio ranges from about 0.01 to about 12, more typically from about 0.05 to about 6, even more typically from about 0.075 to about 3, most typically from about 0.1 to about 0.5.

Suitable non-lipid compounds include molecules having opposite properties on each end of the molecule, for example, a protein, a polypeptide, a polypeptide fragment, a carbohydrate, a dendrimer, a receptor, a hormone, a toxin, and an amphipathic lipid. Suitable non-lipid compounds include, cationic polymers such as, for example, polyethyleneimine, polylysine, polyarginine, and polyomithine and natural DNA-binding proteins of a polycationic nature, such as histones and protamines or analogues or fragments thereof. Additional suitable non-lipid compounds include polyamines such as, for example, spermidine and spermine, and polycations having two or more different positively charged amino acids or basic proteins. Suitable polypeptides include, for example, ID2 and peptides based on it such as, for example ID2-2, ID2-3, ID2-4 (Sperinde et al., J. Gene Med. 3:101 (2001)).

D. Adjuvants

In some embodiments of the present invention, the compositions further comprise an adjuvant. Suitable adjuvants include, for example, the lipids and non-lipid compounds described above, cholera toxin (CT), CT subunit B, CT derivative CTK63, E. coli heat labile enterotoxin (LT), LT derivative LTK63, Al(OH)₃, and polyionic organic acids as defined above and described in e.g., U.S. patent application Ser. No. 60/402,811, filed Aug. 12, 2002 (Bennett et al., “Polyionic Organic Acid Formulations,” Atty. Docket No. 020714-000600), Anderson and Crowle, Infect. Immun. 31(1):413-418 (1981), Roterman et al., J. Physiol. Pharmacol., 44(3):213-32 (1993), Arora and Crowle, J. Reticuloendothel. 24(3):271-86 (1978), and Crowle and May, Infect. Immun. 38(3):932-7 (1982)). Suitable polyionic organic acids include for example, 6,6′-[3,3′-demithyl[1,1′-biphenyl]-4,4′-diyl)bis(azo)bis[4-amino-5-hydroxy-1,3-naphthalene-disulfonic acid] (Evans Blue) and 3,3′-[1,1′biphenyl]-4,4′-diylbis(azo)bis[4-amino-1-naphthalenesulfonic acid] (Congo Red). It will be appreciated by those of skill in the art that the polyionic organic acids may be used for any genetic vaccination method in conjunction with any type of administration. Additional suitable polyionic organic acids are described in, e.g., U.S. patent application Ser. No. ______, filed Aug. 12, 2003 (Bennett et al., “Polyionic Organic Acid Formulations,” Attorney Docket No. 020714-000720).

Other suitable adjuvants include topical immunomodulators such as, members of the imidazoquinoline family such as, for example, imiquimod and resiquimod (see, e.g., Hengge et al., Lancet Infect. Dis. 1(3):189-98 (2001).

Additional suitable adjuvants are commercially available as, for example, additional alum-based adjuvants (e.g., Alhydrogel, Rehydragel, aluminum phosphate, Algammulin); oil based adjuvants (Freund's Incomplete Adjuvant and Complete Adjuvant (Difco Laboratories, Detroit, Mich.), Specol, RIBI, TiterMax, Montanide ISA50 or Seppic MONTANIDE ISA 720); nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres; monophosphoryl lipid A and Quil A. Cytokines, such as GM-CSF or interleukin-2, -7, or -12, are also suitable adjuvants. Hemocyanins (e.g., keyhole limpet hemocyanin) and hemoerythrins may also be used in the invention. Polysaccharide adjuvants such as, for example, chitin, chitosan, and deacetylated chitin are also suitable as adjuvants. Other suitable adjuvants include muramyl dipeptide (MDP, N acetylmuramyl L alanyl D isoglutamine) bacterial peptidoglycans and their derivatives (e.g., threonyl-MDP, and MTPPE). BCG and BCG cell wall skeleton (CWS) may also be used as adjuvants in the invention, with or without trehalose dimycolate. Trehalose dimycolate may be used itself (see, e.g., U.S. Pat. No. 4,579,945). Detoxified endotoxins are also useful as adjuvants alone or in combination with other adjuvants (see, e.g., U.S. Pat. Nos. 4,866,034; 4,435,386; 4,505,899; 4,436,727; 4,436,728; 4,505,900; and 4,520,019. The saponins QS21, QS17, QS7 are also useful as adjuvants (see, e.g., U.S. Pat. No. 5,057,540; EP 0362 279; WO 96/33739; and WO 96/11711). Other suitable adjuvants include Montanide ISA 720 (Seppic, France), SAF (Chiron, Calif., United States), ISCOMS (CSL), MF-59 (Chiron), the SBAS series of adjuvants (e.g., SBAS-2, SBAS-4 or SBAS-6 or variants thereof, available from SmithKline Beecham, Rixensart, Belgium), Detox (Corixa, Hamilton, Mont.), and RC-529 (Corixa, Hamilton, Mont.).

Superantigens are also contemplated for use as adjuvants in the present invention. Superantigens include Staphylococcus exoproteins, such as the α, β, γ and δ enterotoxins from S. aureus and S. epidermidis, and the α, β, γ and δ E. coli exotoxins. Common Staphylococcus enterotoxins are known as staphylococcal enterotoxin A (SEA) and staphylococcal enterotoxin B (SEB), with enterotoxins through E (SEE) being described (Rott et al., 1992). Streptococcus pyogenes B (SEB), Clostridium perfringens enterotoxin (Bowness et al., 1992), cytoplasmic membrane-associated protein (CAP) from S. pyogenes (Sato et al., 1994) and toxic shock syndrome toxin 1 (TSST 1) from S. aureus (Schwab et al., 1993) are further useful superantigens.

Within the pharmaceutical compositions provided herein, the adjuvant composition can be designed to induce, e.g., an immune response predominantly of the Th1 or Th2 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following retroductal introduction of a composition comprising an immunogenic polypeptide as provided herein, an immune response that includes Th1- and Th2-type responses will typically be elicited.

Pharmaceutical compositions within the scope of the present invention may also contain other compounds, which may be biologically active or inactive. Polypeptides may, but need not, be conjugated to other macromolecules as described, for example, within U.S. Pat. Nos. 4,372,945 and 4,474,757. Pharmaceutical compositions may generally be used for prophylactic and therapeutic purposes.

A pharmaceutical composition or vaccine may contain a polynucleotide encoding an immunogenic polypeptide. Such a polynucleotide may comprise DNA, RNA, a modified nucleic acid or a DNA/RNA hybrid. As noted above, a polynucleotide may be present within any of a variety of delivery systems known to those of ordinary skill in the art, including nucleic acid expression systems and viral expression systems. Numerous gene delivery techniques are well known in the art, such as those described by Rolland (1998) Crit. Rev. Therap. Drug Carrier Systems 15:143-198, and references cited therein. Appropriate nucleic acid expression systems contain the necessary DNA sequences for expression in the patient (such as a suitable promoter and terminating signal).

It will be apparent that a vaccine may contain pharmaceutically acceptable salts of the polynucleotides encoding immunogenic polypeptides. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Any suitable carrier known to those of ordinary skill in the art may be employed in the pharmaceutical compositions of this invention. Suitable carriers include, for example, water, saline, alcohol, a fat, a wax, a buffer, a solid carrier, such as mannitol, lactose, starch, magnesium stearate, sodium saccharine, talcum, cellulose, glucose, sucrose, and magnesium carbonate, or biodegradable microspheres (e.g., polylactate polyglycolate). Suitable biodegradable microspheres are disclosed, for example, in U.S. Pat. Nos. 4,897,268; 5,075,109; 5,928,647; 5,811,128; 5,820,883. The immunogenic polypeptide may be encapsulated within the biodegradable microsphere or associated with the surface of the microsphere.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule or sponge that effects a slow release of compound following administration). Such formulations may generally be prepared using well known technology (see, e.g., Coombes et al. (1996) Vaccine 14:1429-1438). Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254; and PCT applications WO 94/20078; WO/94/23701; and WO 96/06638). The amount of active compound contained within a sustained release formulation depends upon the site of implantation, the rate and expected duration of release and the nature of the condition to be treated or prevented.

The pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

### III. Administration of the Compositions of the Present Invention

According to the methods of the present invention, a composition comprising a nucleic acid encoding an immunogenic polypeptide is retroductally introduced into the lumen of a salivary gland duct. The nucleic acid may be a in a vector as described above or may be “naked” as described in, e.g., Ulmer et al. (1993) Science 259:1745-1749 and reviewed by Cohen (1993) Science 259:1691-1692. The composition may be introduced alone or with an adjuvant as described above. In some embodiments of the present invention, the adjuvant is administered at the same time as the composition. In other with embodiments of the present invention, the adjuvant is administered after the composition, e.g., 6, 12, 18, 24, 36, 48, 60, or 72 hours after administration of the composition.

Suitable methods of retroductal introduction of the composition to the salivary gland duct include, for example, cannulation or injection of the composition into the salivary gland duct using a syringe, cannula, catheter, or shunt. The type of syringe, cannula, catheter, or shunt used is not a critical part of the invention. One of skill in the art will appreciate that multiple types of syringes, cannulas, catheters, or shunts may be used to administer compositions according to the methods of the present invention.

Retroductal delivery of the composition using the methods of the present invention may be via gravity or an assisted delivery system. Suitable assisted delivery systems include controlled release pumps, time release pumps, osmotic pumps, and infusion pumps. The particular delivery system or device is not a critical aspect of the invention. One of skill in the art will appreciate that multiple types of assisted delivery systems may be used to deliver compositions according to the methods of the present invention. Suitable delivery systems and devices are described in U.S. Pat. Nos. 5,492,534, 5,562,654, 5,637,095, 5,672,167, and 5,755,691. One of skill in the art will also appreciate that the infusion rate for delivery of the composition may be varied. Suitable infusion rates may be from about 0.005 ml/min to about 1 ml/minute, preferably from about 0.01 ml/min to about 0.8 ml/min., more preferably from about 0.025 ml/min. to about 0.6 ml/min. It is particularly preferred that the infusion rate is about 0.05 ml/min.

### IV. Immune Response to Immunogenic Polypeptides

A. Detection of an Immune Response to Immunogenic Polypeptides

In one embodiment of the present invention, polynucleotides that encode immunogenic polypeptides are used to generate an immune response (i.e., a mucosal, humoral, or cell-mediated immune response) to antigens, such as, for example, cancer antigens, bacterial antigens, viral antigens, fungal antigens, or parasite antigens. Representative examples of cancer antigens include antigens expressed, for example, in colon cancer, stomach cancer, liver cancer, pancreatic cancer, lung cancer, ovarian cancer, prostate cancer, breast cancer, skin cancer (e.g., melanoma), leukemia, lymphoma, or myeloma. Exemplary cancer antigens include, for example, HPV L1, HPV L2, HPV E1, HPV E2, PSA, placental alkaline phosphatase, AFP, BRCA1, Her2/neu, CA 15-3, CA 19-9, CA-125, CEA, hCG, urokinase-type plasminogen activator (uPA), plasminogen activator inhibitor and MAGE-1. Bacterial antigens may be derived from, for example, Staphylococcus aureus, Staphylococcus epidermis, Helicobacter pylori, Streptococcus bovis, Streptococcus pyogenes, Streptococcus pneumoniae, Listeria monocytogenes, Mycobacterium tuberculosis, Mycobacterium leprae, Corynebacterium diphtheriae, Borrelia burgdorferi, Bacillus anthracis, Bacillus cereus, Clostridium botulinum, Clostridium difficile, Salmonella typhi, Vibrio chloerae, Haemophilus influenzae, Bordetella pertussis, Yersinia pestis, Neisseria gonorrhoeae, Treponema pallidum, Mycoplasm sp., Neisseria meningitidis, Legionella pneumophila, Rickettsia typhi, Chlamydia trachomatis, and Shigella dysenteriae. Viral antigens may be derived from, for example, human immunodeficiency virus, human papilloma virus, Epstein Barr virus, herpes simplex virus, human herpes virus, rhinoviruses, cocksackieviruses, enteroviruses, hepatitis A, hepatitis B, hepatitis C, and hepatitis E, rotaviruses, mumps virus, rubella virus, measles virus, poliovirus, smallpox virus, influenza virus, rabies virus, and Variella-zoster virus. Fungal antigens may be derived from, for example, Tinea pedis, Tinea corporus, Tinea cruris, Tinea unguium, Cladosporium carionii, Coccidioides immitis, Candida sp., Aspergillus fumigatus, and Pneumocystis carinii. Parasite antigens may be derived from, for example, Giardia lamblia, Leishmania sp., Trypanosoma sp., Trichomonas sp., Plasmodium sp., and Schistosoma sp.

An immune response to the immunogenic polypeptides can be long-lived and can be detected long after immunization, regardless of whether the protein is present or absent in the body at the time of testing. An immune response to the immunogenic polypeptide can be detected by examining for the presence or absence, or enhancement, of specific activation of CD4⁺ or CD8⁺ T cells or by antibodies. For instance, T cells isolated from an immunized individual by routine techniques (e.g., by Ficoll/Hypaque density gradient centrifugation of peripheral blood lymphocytes) are incubated with the immunogenic polypeptide. For example, T cells may be incubated in vitro for 2-9 days (typically 4 days) at 37° C. with an immunogenic polypeptide (typically, about 0.2 to about 5 μg/ml). It may be desirable to incubate another aliquot of a T cell sample in the absence of the immunogenic polypeptide to serve as a control.

Specific activation of CD4⁺ or CD8⁺ T cells associated with a mucosal, humoral, or cell-mediated immune response may be detected in a variety of ways. Methods for detecting specific T cell activation include, but are not limited to, detecting the proliferation of T cells, the production of cytokines (e.g., lymphokines), or the generation of cytolytic activity (i.e., generation of cytotoxic T cells specific for the immunogenic polypeptide). For CD4⁺ T cells, a preferred method for detecting specific T cell activation is the detection of the proliferation of T cells. For CD8⁺ T cells, a preferred method for detecting specific T cell activation is the detection of the generation of cytolytic activity using ⁵¹Cr release assays (see, e.g., Brossart and Bevan, Blood 90(4): 1594-1599 (1997) and Lenz et al., J. Exp. Med . 192(8): 1135-1142 (2000)).

Detection of the proliferation of T cells may be accomplished by a variety of known techniques. For example, T cell proliferation can be detected by measuring the rate of DNA synthesis. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca²⁺ flux, or dye uptake, such as 3-(4,5-dimethylthiazol-2-yl)-2,5-diphenyltetrazolium. Alternatively, synthesis of lymphokines (e.g., interferon-gamma) can be measured or the relative number of T cells that can respond to the immunogenic polypeptide may be quantified.

Humoral immune responses, including mucosal humoral responses can be detected using immunoassays known in the art. Suitable immunoassays include the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “western blot” method of Gordon et al. (U.S. Pat. No. 4,452,901); immunoprecipitation of labeled ligand (Brown et al. (1980) J. Biol. Chem. 255:4980-4983); enzyme-linked immunosorbent assays (ELISA) as described, for example, by Raines et al. (1982) J. Biol. Chem. 257:5154-5160; immunocytochemical techniques, including the use of fluorochromes (Brooks et al. (1980) Clin. Exp. Immunol. 39:477); and neutralization of activity (Bowen-Pope et al. (1984) Proc. Natl. Acad. Sci. USA 81:2396-2400). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

Monoclonal antibodies to the immunogenic peptides can be generated using methods known in the art (see, e.g., Kohler and Milstein, Nature 256: 495-497 (1975) and Harlow and Lane, ANTIBODIES, A LABORATORY MANUAL, Cold Spring Harbor Publication, New York (1999)). Generation of monoclonal antibodies has been previously described and can be accomplished by any means known in the art. (Buhring et al. in Hybridoma 1991, Vol. 10, No. 1, pp. 77-78). For example, an animal such as a guinea pig or rat, preferably a mouse is immunized with an immunogenic polypeptide, the antibody-producing cells, preferably splenic lymphocytes, are collected and fused to a stable, immortalized cell line, preferably a myeloma cell line, to produce hybridoma cells which are then isolated and cloned. (U.S. Pat. No. 6,156,882).

Binding of a monoclonal antibody to the immunogenic polypeptide presented on the surface the transfected cell may be detected by direct (in the case of labeled antibodies) or indirect (in the case of unlabeled antibodies) methods known in the art and described in e.g., Ausubel et al., supra and Harlow and Lane, 1999, supra. For example, flow cytometry or enzyme linked immunosorbent assays can be used to detect MHC Class II/peptide complexes or MHC Class I/peptide complexes on the surface of antigen presenting cells.

The particular label or detectable group used in the assay is not a critical aspect of the invention, as long as it does not significantly interfere with the specific binding of the antibody to the immunogenic polypeptide. The detectable group can be any material having a detectable physical or chemical property. Thus, a label is any composition detectable by spectroscopic, photochemical, biochemical, electrical, optical or chemical means. A wide variety of labels may be used, with the choice of label depending on sensitivity required, ease of conjugation with the compound, stability requirements, available instrumentation, and disposal provisions. Useful labels in the present invention include magnetic beads (e.g., DYNABEADSTM), fluorescent dyes (e.g., fluorescein isothiocyanate, Texas red, rhodamine, and the like), radiolabels (e.g., ³H, ¹²⁵I, ³⁵S, ¹⁴C, or ³²P), and colorimetric labels such as colloidal gold or colored glass or plastic beads (e.g., polystyrene, polypropylene, latex, etc.).

The molecules can be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

For detection purposes, immunogenic polypeptides (i.e., antigens) may either be labeled or unlabeled. When unlabeled, immunogenic polypeptides find use in agglutination assays. In addition, unlabeled immunogenic polypeptides can be used in combination with labeled molecules that are reactive with immunocomplexes, or in combination with labeled antibodies (second antibodies) that are reactive with the antibody directed against the immunogenic polypeptide. Alternatively, the immunogenic polypeptide can be directly labeled. Where it is labeled, the reporter group can include, e.g., radioisotopes, fluorophores, enzymes, luminescers, dye particles and the like. These and other labels are well known in the art and are described, for example, in U.S. Pat. Nos. 3,766,162; 3,791,932; 3,817,837; 3,996,345; and 4,233,402.

Typically in an ELISA, the immunogenic polypeptide is adsorbed to the surface of a microtiter well. Residual protein-binding sites on the surface are then blocked with an appropriate agent, such as bovine serum albumin (BSA), heat-inactivated normal goat serum (NGS), or BLOTTO (buffered solution of nonfat dry milk which also contains a preservative, salts, and an antifoaming agent). The well is then incubated with a sample (e.g., a biological sample from the subject to whom the composition comprising a nucleic acid encoding the immunogenic polypeptide was administered) suspected of containing specific antibody. The sample can be applied neat, or, more often, it can be diluted, usually in a buffered solution which contains a small amount (0.1%-5.0% by weight) of protein, such as BSA, NGS, or BLOTTO. After incubating for a sufficient length of time to allow specific binding to occur, the well is washed to remove unbound protein and then incubated with an anti-species specific immunoglobulin antibody labeled with a reporter group. The reporter group can be chosen from a variety of enzymes, including, e.g., horseradish peroxidase, beta-galactosidase, alkaline phosphatase, and glucose oxidase. Sufficient time is allowed for specific binding to occur, then the well is again washed to remove unbound conjugate, and the substrate for the enzyme is added. Color is allowed to develop and the optical density of the contents of the well is determined visually or instrumentally.

In one embodiment of this aspect of the present invention, a reporter group is bound to the immunogenic polypeptide of interest. The step of detecting immunocomplexes involves removing substantially any unbound immunogenic polypeptide and then detecting the presence or absence of the reporter group. In another embodiment, a reporter group is bound to a second antibody capable of binding to the antibodies specific for immunogenic polypeptide. The step of detecting immunocomplexes involves (a) removing substantially any unbound antibody, (b) adding the second antibody, (c) removing substantially any unbound second antibody and then (d) detecting the presence or absence of the reporter group. Where the antibody specific for the immunogenic polypeptide of interest is derived from a human, the second antibody is an anti-human antibody. In a third embodiment for detecting immunocomplexes, a reporter group is bound to a molecule capable of binding to the immunocomplexes. The step of detecting involves (a) adding the molecule, (b) removing substantially any unbound molecule, and then (c) detecting the presence or absence of the reporter group. An example of a molecule capable of binding to the immunocomplexes is protein A.

It will be evident to one skilled in the art that a variety of methods for detecting the immunocomplexes may be used within the present invention. Reporter groups suitable for use in any of the methods include, e.g., radioisotopes, fluorophores, enzymes, luminescers, and dye particles.

### V. Disease Prevention or Therapy

One aspect of the present invention involves using the immunogenic compositions described herein to elicit an antigen specific immune response from a subject or patient with a disease such as, for example, a viral infection, bacterial infection, a parasitic infection, a fungal infection, or cancer. As used herein, a “subject” or a “patient” refers to any warm-blooded animal, such as, for example, a rodent, a feline, a canine, or a primate, preferably a human. The immunogenic compositions may be used to treat at any stage of the disease, i.e., at the pre-cancer, cancer, or metastatic stages, or to prevent disease.

As an illustrative example, the compositions described herein may be used for immunotherapy (i.e., prevention or treatment) of cancer, such as breast, ovarian, colon, lung and prostate cancer. Within such methods, pharmaceutical compositions are typically administered to a patient. A patient may or may not be afflicted with cancer. Accordingly, the above pharmaceutical compositions may be used to prevent the development of a cancer or to treat a patient afflicted with a cancer. A cancer may be diagnosed using criteria generally accepted in the art, including the presence of a malignant tumor. Pharmaceutical compositions may be administered either prior to or following surgical removal of primary tumors and/or treatment such as administration of radiotherapy or conventional chemotherapeutic drugs.

In a further illustrative example the compositions described herein may be used for immunotherapy (i.e., prevention or treatment) of bacterial, viral, fungal, or parasitic diseases and disorders. Within such methods, pharmaceutical compositions are typically administered to a patient. A patient may or may not be afflicted with the disease or disorder. Accordingly, the above pharmaceutical compositions may be used to prevent the development of a particular disease or disorder or to treat a patient afflicted with a disease or disorder. A disease or disorder may be diagnosed using criteria generally accepted in the art.

Immunotherapy is typically active immunotherapy, in which treatment relies on the in vivo stimulation of the endogenous host immune system to react against, e.g., tumors or bacterially or virally infected cells, with the administration of immune response-modifying agents (compositions comprising nucleic acids encoding immunogenic polypeptides as provided herein).

Frequency of administration of the prophylactic or therapeutic compositions described herein, as well as dosage, will vary from individual to individual, and may be readily established using standard techniques. Often between 1 and 10 doses may be administered over a 52 week period. Typically 6 doses are administered, at intervals of 1 month, more typically, 2-3 doses are administered every 2-3 months. Booster vaccinations may be given periodically thereafter. Alternate protocols may be appropriate for individual patients and particular diseases and disorders. A suitable dose is an amount of a compound that, when administered as described above, is capable of promoting, e.g., an anti-tumor, an anti-viral, or an antibacterial, immune response, and is at least 10-50% above the basal (i.e., untreated) level. Such response can be monitored by measuring the anti-tumor antibodies in a patient or by vaccine-dependent generation of cytolytic effector cells capable of killing, e.g., the patient's tumor cells, the patient's virally infected cells, or the patient's bacterially infected cells in vitro. Such vaccines should also be capable of causing an immune response that leads to an improved clinical outcome (e.g., more frequent remissions, complete or partial or longer disease-free survival) in vaccinated patients as compared to non-vaccinated patients. Typically, the amount of the nucleic acid encoding an immunogenic polypeptide present in a dose ranges from about 1 82 g to 5 mg, preferably 100 μg to 5 mg, and most preferably 5 μg to 300 μg per kg of host. Suitable dose sizes will vary with the size of the patient, but will typically range from about 0.01 ml to about 10 ml, more typically from about 0.025 to about 7.5 ml, most typically from about 0.05 to about 5 ml. Those of skill in the art will appreciate that the dose size may be adjusted based on the particular patient or the particular disease or disorder being treated.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Such immune responses may generally be evaluated using standard proliferation, cytotoxicity or cytokine assays described above, which may be performed using samples obtained from a patient before and after treatment.

For example, detection of immunocomplexes formed between immunogenic polypeptides and antibodies in body fluid which are specific for immunogenic polypeptides may be used to monitor the effectiveness of therapy, which involves a particular immunogenic polypeptide, for a disease or disorder in which the immunogenic polypeptide is associated. Samples of body fluid taken from an individual prior to and subsequent to initiation of therapy may be analyzed for the immunocomplexes by the methodologies described above. Briefly, the number of immunocomplexes detected in both samples are compared. A substantial change in the number of immunocomplexes in the second sample (post-therapy initiation) relative to the first sample (pre-therapy) reflects successful therapy.

All publications and patent applications cited in this specification are herein incorporated by reference as if each individual publication or patent application were specifically and individually indicated to be incorporated by reference.

### EXAMPLES

### Example 1

### Materials and Methods

Animals and plasmids: Sprague-Dawley rats were obtained from Harlan (Indianapolis, Ind.). Plasmid encoding the human growth hormone (hGH) gene was originally obtained from M. German (see, e.g., Goldfine et al., Nat. Biotechnol. 15(13):1378-1382 (1997) and placed under the control of the CMV promoter to generate pFOXCMVhuGH-G3. The DNA encoding HIV envelope (gp120) was obtained from the NIH AIDS Research and Reference Reagent Program (see, e.g., Andre et al., J. Virol. 72(2):1497-1503 (1998) and placed under control of the CMV promoter. Plasmids were purified under endotoxin-reduced conditions using Qiagen's Gigaprep kits. Endotoxin less than 100 E.U./mg DNA as measured by clot LAL assay (Charles River's Endosafe).

DNA and protein vaccination: Retroductal DNA delivery to the salivary glands has been described previously (see, e.g., Goldfine et al., 1997, supra and U.S. Pat. No. 6,372,722). Briefly, after anesthesia polyethylene (PE) 10 tubes were inserted into the left and right duct openings of the submandibular glands. Aqueous solutions of DNA were instilled by retrograde infusion using a syringe pump. Each infusion contained either 88 μg or 175 μg in 200 μl water per gland, depending on the experiment. In order to enhance expression, DNA was co-formulated in one experiment with 3.6 mM Zn in water and co-formulated in two experiments using a lipid and 0.125 mM Zn (Lipid/Zn) formulation (see, e.g., U.S. Pat. No. 6,372,722 and Pichon et al., J. Gene Med. (2002), available at www3.interscience.wiley.com/cgi-bin/abstract/94518178/START). The lipid was a 3:1 ratio of N,N,N′,N′-tetramethyl-N,N′-bis(2-hydroxyethyl)-2,3-di(oleoyloxy)-1,4-butanediaminium iodide: dioleoylphosphatidylethanolamine (DOHBD:DOPE) (see, e.g., U.S. Pat. No. 5,527,928). I.m. DNA vaccination was performed by bilateral injection of either 88 or 175 μg of DNA and saline to the quadriceps of rats. For protein vaccination, 15 μg protein was added to sterile saline and mixed in a 50/50 mixture with Complete Freund's Adjuvant (CFA) to form an emulsion. 100 μl of the emulsion was delivered by subcutaneous injection.

ELISA Assays: To measure gene expression with the salivary glands, the glands were removed by dissection, added to phosphate buffered saline, and homogenized to make lysates. hGH protein was measured by commercial ELISA kit (Roche, Indianapolis, Ind.). For specific IgA and IgG measurements, microtiter plates were coated in 1× carbonate buffer with 1.0 μg/ml protein, either hGH (Fitzgerald, Concord, Mass.) or gp120 (Fitzgerald). Plates were incubated overnight at 4° C. in a humidified chamber, then the plates were blocked in PBS+0.05% Tween 20 (PBST)+1% Bovine serum albumin (BSA) solution for 1 hour before washing. Plasma samples were serially diluted in PBST. After a two hour incubation, plates were washed with PBST at least 6 times. Antibodies were then added, either anti-rat IgG-Horse Radish Peroxidase (HRP) (Sigma) or anti-rat IgA-HRP (Bethyl labs. Montgomery, Tex.), or anti-rat secretory component-HRP (Bethyl labs) each at 1:2000 dilution. Plates were washed at least 6 times after a 1 hour incubation. Antigen specific rat antibodies were detected with 3,3′,5,5′-tetramethyl-Benzidine (TMB) substrate (Dako) using a microplate reader. Antibody titers were reported as the reciprocal dilution giving an absorbance value greater than 2 standard deviations times the average background. O.D. values were reported using equivalent amounts of IgA in the assay. For salivary IgA, saliva was collected by cannulating the salivary duct followed by 1.0 mg/ml pilocarpine injection s.c. Total IgA was measured by coating ELISA plates with mouse anti-rat IgA at 5 μg/ml (Biosource) and detecting with mouse anti-rat light chain-HRP at 1:1000 dilution (Serotec, Raleigh, N.C.) using a rat IgA standard (Biosource).

ELISPOTS: To assess the numbers of antibody secreting cells (ASC), an enzyme-linked immunospot (ELISpot) was performed as described (see, e.g., Yamamoto et al., J. Immunol. 161(8):4115-4121 (1998)). Millipore HA plates were coated with 50 μl/well of 10 μg/ml gp120 protein under sterile conditions and incubated overnight at 4° C. in a humidified chamber. The next day, the plate was washed with sterile PBST and blocked with w/RPMI+5% fetal bovine serum (FBS) for 1 hour at 37° C. while shaking. Peyer's patches were excised from the serosal side of the small intestine and then pushed through a sterile nylon strainer to isolate single cells (see, e.g., Lycke et al.J. Immunol. 163(2):913-919 (1999)). The cells were washed and resuspended in RPMI+5% FBS. 5.0×10⁵ cells per well were added in triplicates for each individually prepared sample. The plate was washed 3× with PBST and 3× with RPMI+5% FBS. The next day, the cells were removed by flipping the plate. The plate was then washed 10× with PBST. Antibodies were diluted in PBST+1% BSA at desired concentrations. For anti-rat IgA-biotin, 1:100 dilution was used. 100 μl of diluted antibody was added to each well and the plate was incubated at RT for 4 hours. The plate was then washed 10× with PBST. Strepavidin-HRP was added at 4 mg/mL, 100 μl per well and incubated at RT for 1 hour while shaking. The plate was then washed 5× with PBST, washed 5× with PBS by hand, and then flipped dry by tapping the plate. AEC (3-amino-9-ethylcarbonate) solution (Sigma) was prepared according the manufactures recommendations and 100 μl per well was added. The plate was developed for 2.5 to 4 minutes then immediately rinsed with deionized water. The plate was kept inverted in the dark until dry (overnight). The numbers of spots (ASC) were read “blind” by Zellnet Consulting (New York, N.Y.).

T cell assays: Splenocytes were isolated by breaking the spleen capsula and then pushing the cells through a sterile strainer into NH₄Cl₂ lysis solution. After a 5 minute incubation, the cells were washed in IMDM media (Invitrogen, San Diego, Calif.)+10% serum and counted. For γ-interferon (γ-IFN) assays, 4×10⁶ cells/ml were cultured in IMDM media with mouse IL-2 (BioSource, Camarillo, Calif.) at 10 ng/ml and rat IL-4 (BioSource) at 50 ng/ml for 4 days. Some samples were cultured with gp120 at either 0.2 or 1 μg/ml. Cells were cultured in triplicate in 96 well plates with 100 μl per well. The supernate was used in an ELISA for rat γ-IFN (R & D systems, Minneapolis, Minn.). Intracellular cytokine stained was performed as previously described (see, e.g., Edelmann and Wilson, J. Virol. 75(2):612-621 (2001)). Briefly, cells were cultured with 0.2 μg/ml gp120 in IMDM+10% serum and 1.0×10⁻⁵ M β-mercaptoethanol. The next day, the cells were treated with brefeldin A for 4 hours to block protein transport. The cells were then stained for extracellular markers of CD4 and CD8, fixed in 2% paraformaldehyde, and permeabilized with 0.5% saponin in PBS. Antibodies that recognize γ-IFN or an isotype control were added and incubated for 20 minutes on ice. The cells were then washed twice with 0.5% saponin in PBS to remove unbound antibody. The cells were analyzed by a flow cytometer (BD FACsCalibur) to determine the percentage of cells that were γ-IFN positive, of either CD4 or CD8 T cell subtypes. Anti-CD4 and anti-CD8 antibodies were obtained from BD Pharmingen (San Diego, Calif.)

### Example 2

### Systemic IgG and IgA Responses are Elicited by Salivary Gland Genetic Vaccination

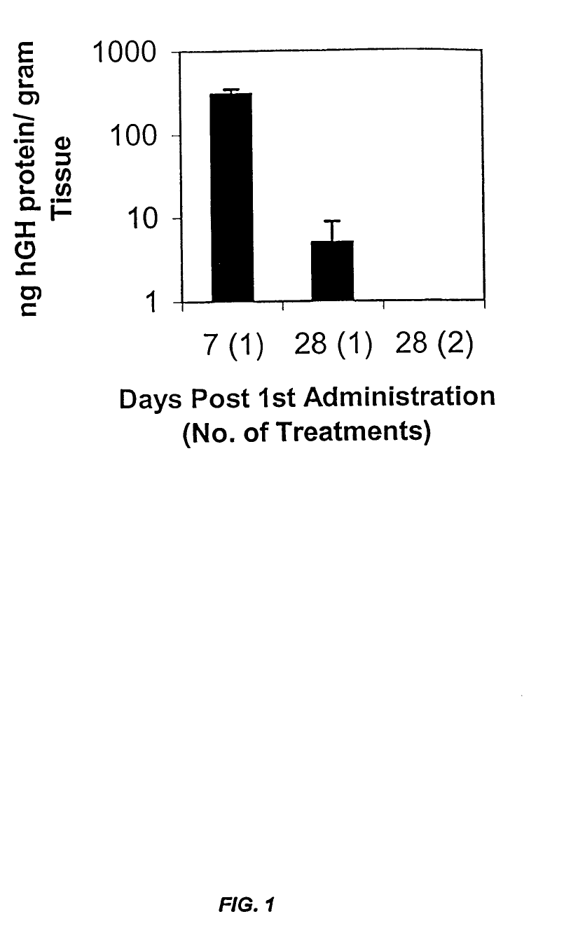

Retroductal delivery of gene vectors to the salivary glands has been described as an efficient method of both local and systemic protein delivery. When plasmid DNA encoding human growth hormone (hGH) was delivered to the salivary glands (submandibular) of rats, significant levels of hGH protein was detected in the glands 7 days after DNA administration (FIG. 1). The amount of protein present decreased substantially over 28 days, and that retreatment of the gland with plasmid did not restore expression (FIG. 1).

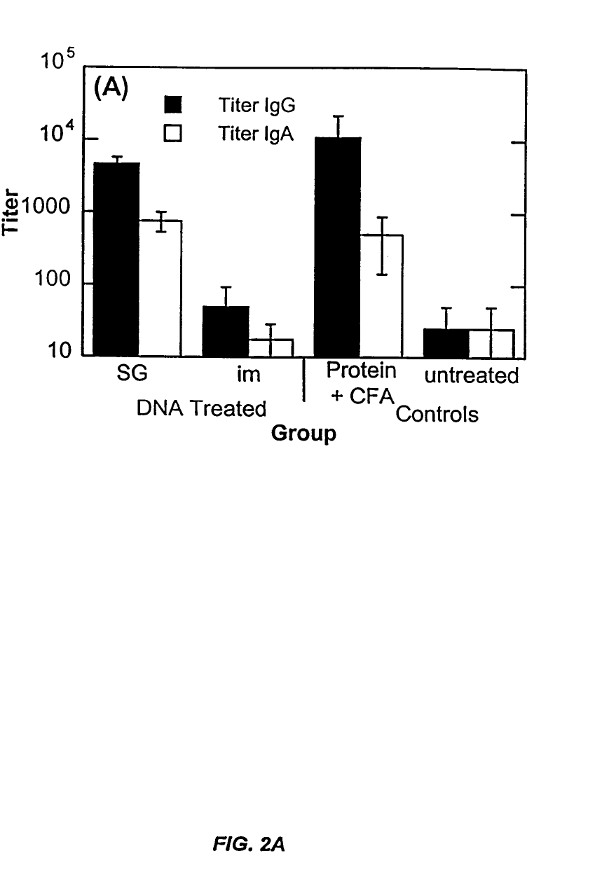

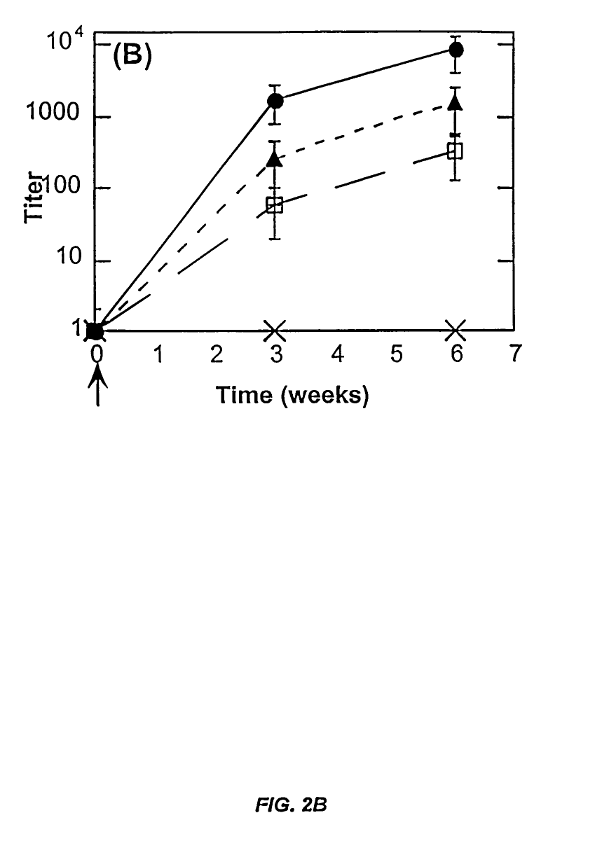

It has been shown previously that injection of hGH protein induces an immune responses in rats (see, e.g., Bennett et al, Mol. Biol. Med. 7:471 (1990)). To test the hypothesis that the inability to retreat salivary glands with hGH plasmid results from a strong adaptive immune response to the encoded protein, plasmid DNA encoding hGH protein was delivered to the salivary glands by retroductal delivery. For comparison, an equal amount of hGH DNA was injected i.m. and hGH protein, formulated with complete Freund's adjuvant (CFA), was injected s.c. Plasma from the animals was collected 3 weeks after DNA or protein treatment. The plasma was analyzed by ELISA for anti-hGH IgG antibody titers. A significant humoral response to hGH protein was observed following retroductal salivary gland genetic vaccination (RSGV); a single delivery of plasmid encoding hGH to rat submandibular salivary glands in a simple water buffer induced anti-hGH titers greater than 4.0×10³ in 3 weeks (FIG. 2). The mean IgG titers obtained following SG delivery were found to be approximately 46 fold greater than titers following i.m. injection on a per μg of DNA basis (p=0.026 by Mann-Whitney U). The disparity was even greater when the titers of circulating IgA (FIG. 2)were compared; an 85 fold greater IgA response in the SG treated animals was observed compared to animals who received injection to the muscle (p=0.021 by Mann-Whitney U). In comparison to protein inoculation with hGH protein plus CFA, RSGV yielded lower IgG titers, and nearly equivalent IgA titers. These results demonstrate that RSGV induces potent antibody responses.

The discrepancy between i.m. injection and RSGV in terms of antibody titers prompted further investigation. One explanation for the superior performance of the RSGV is that inoculation in the oral mucosa, not necessarily the salivary glands, is more effective than muscle. To determine whether the salivary gland itself contributed to robust immune responses, or whether the effect could be achieved by injection into tissue surrounding the gland, rats were inoculated with equal amounts of hGH plasmid DNA by injection into the submucosa or muscle, or by retroductal perfusion of the salivary gland. The titers of circulating anti-hGH antibodies were measured by ELISA at 3 and 6 weeks post treatment. RSGV outperformed both i.m. and submucosal injection as measured by the systemic IgG (FIG. 2B) and IgA titers. Thus, injection into the submucosa was insufficient to generate the magnitude of immune response observed with RSGV.

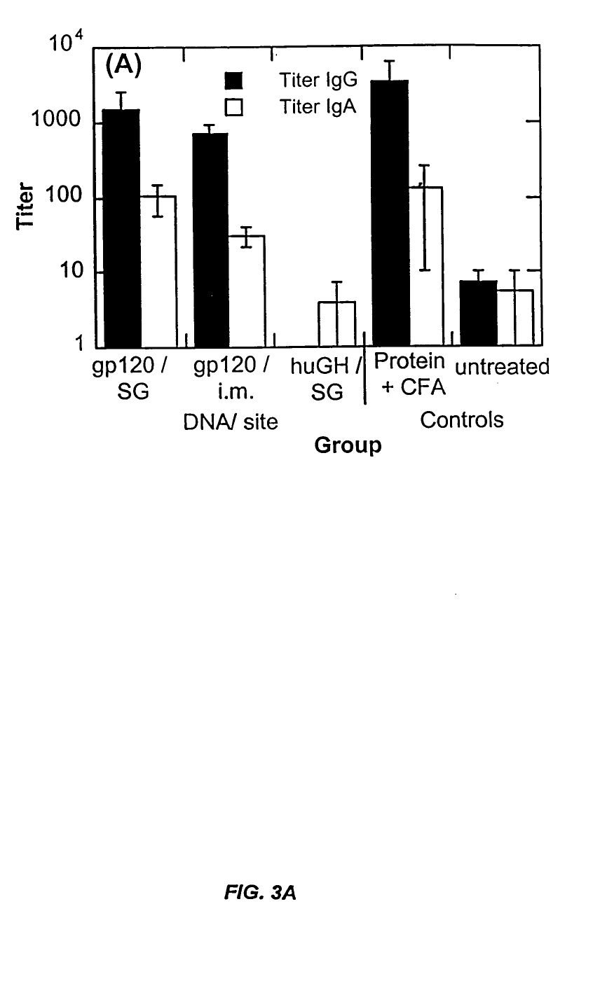

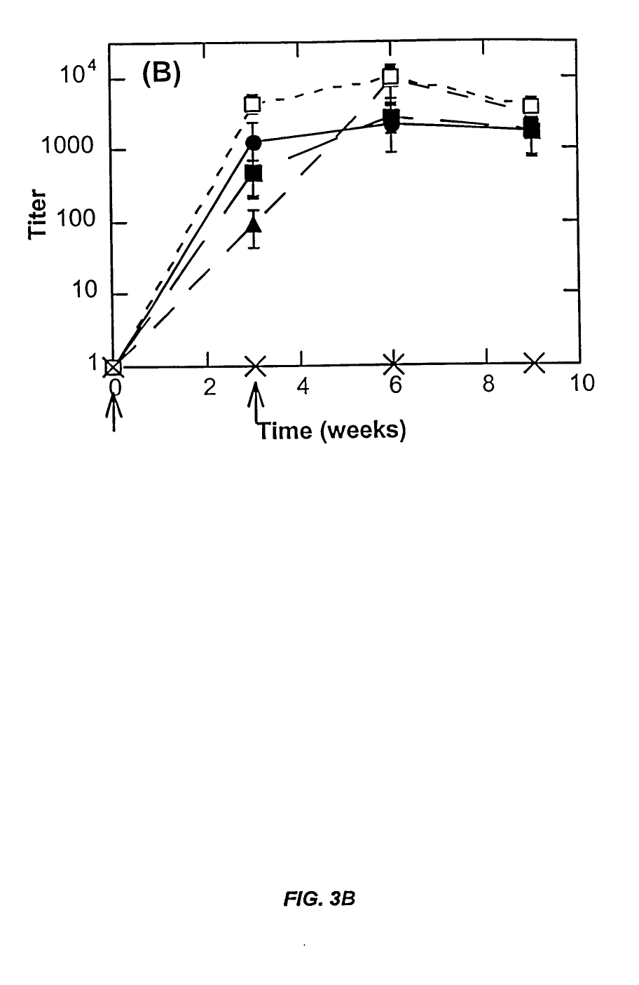

To determine whether RSGV was applicable to other antigens, a disease-related antigen, the HIV envelope protein gp120 was tested. Rats were treated with equal amounts of gp120 DNA by RSGV or by i.m. injection. Plasma samples were assayed 6 weeks after the first treatment, two weeks after the last DNA treatment for a total of two administrations. Rats were also treated with gp120 protein plus CFA as a control. The plasma IgG and IgA responses to gp120 were typically better with RSGV than with i.m., although the IgG titers were stastically insignificant in this experiment (FIG. 3A). The anti-gp 120 IgG and IgA titers resulting from protein plus CFA were similar to RSGV, with the average IgG slightly higher with protein. The temporal pattern of IgG production was also evaluated (FIG. 3B). The antibody responses to gp 120 protein at 9 weeks reached an average IgG titer above 8.0×10³.

### Example 3

### Both CD8 and CD4 T Cells Responses are Induced Upon Salivary Gland Vaccination

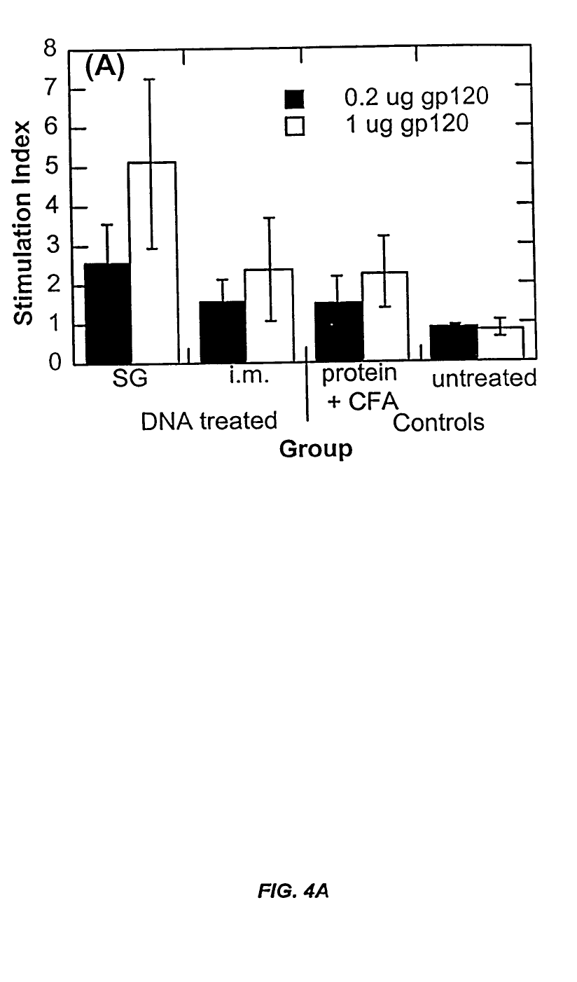

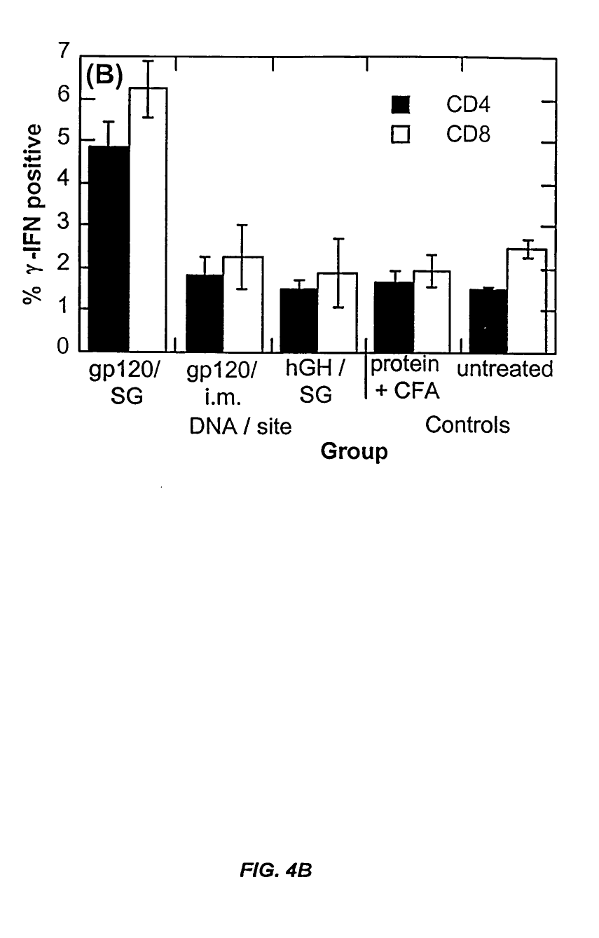

Systemic cellular immune responses were also evaluated. DNA vaccination by i.m. injection has been shown to produce a predominant Th1 response (see, e.g., McCluskie et al., Mol. Med. 5: 287 (1999)). To quantify the ability of RSGV and i.m. to elicit antigen specific immune responses, γ-IFN production was measured from splenocytes isolated from DNA vaccinated animals. 175 μg HIV gp120 DNA was delivered by RSGV or i.m. injection on weeks 0,4, and 8 and harvested on week 9. Splenocytes from these rats were cultured for 4 days with either 0, 0.2, or 1 μg/ml gp120 protein. RSGV resulted in significantly more γ-IFN secreted by cultured splenocytes than either i.m. DNA or s.c. protein, and the γ-IFN produced increased with increasing amounts of antigen (FIG. 4A). This suggests that a high proportion of T cells recognize the antigen gp120 in the RSGV animals. T cell activity was examined using an intracellular γ-IFN flow cytometry assay. This assay provided additional information as to which T cells could be contributing to the secreted γ-IFN shown above. Animals were vaccinated 3 times, as described previously, and harvested one week after the last DNA inoculation. After overnight culture with 0.2 μg/ml gp120 antigen, cells were stained for CD4 and CD8 surface markers as well as for intracellular γ-IFN. γ-IFN intracellular responses on CD4+T cells demonstrates both T helper cell polarization and cell proliferation (see, e.g., Bird et al., Immunity 9:229 (1998)). A high proportion of T helper cells (CD4+CD8−) were γ-IFN positive (FIG. 4B). Further, the proportion of γ-IFN+T helper cells was higher with RSGV than with i.m. DNA vaccination (FIG. 4B). CTL activity can be measured by determining the proportion of CD8+T cells that are intracellular γ-IFN positive. This assay correlates well with CTL function, as measured by ⁵¹Cr release assay (see, e.g., Edelman and Wilson, 2001, supra). The frequency of CTL (CD4−CD8+) that were γ-IFN+ was on average 6% when DNA was delivered to the salivary gland via the duct, with 6 out of 6 animals scoring higher than untreated controls. In contrast, only 2 out of 6 i.m. treated animals were weakly positive (FIG. 4). Thus, salivary gland vaccination promoted both systemic T cell and antibody responses.

### Example 4

### Mucosal Immune Responses are Significantly Heightened by Salivary Gland DNA Vaccination

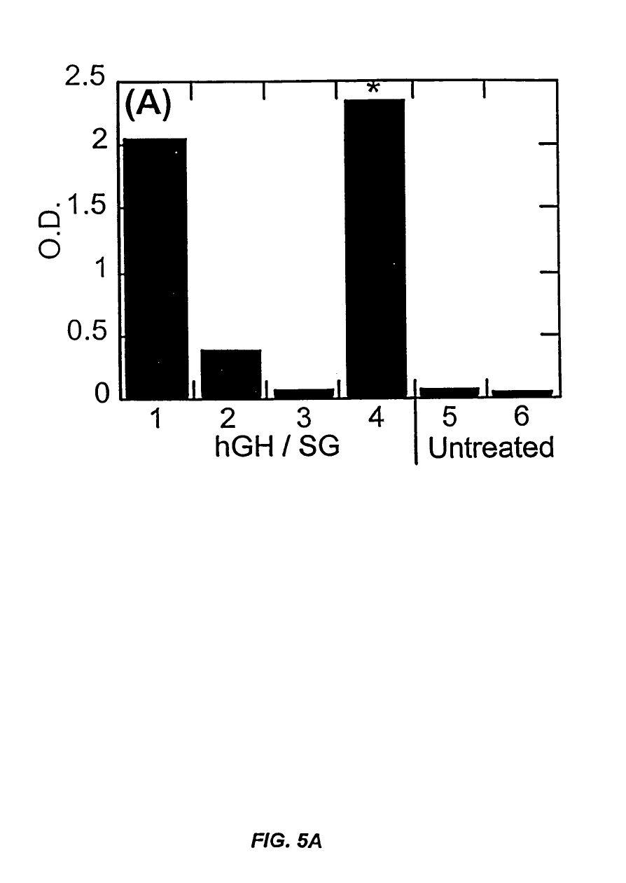

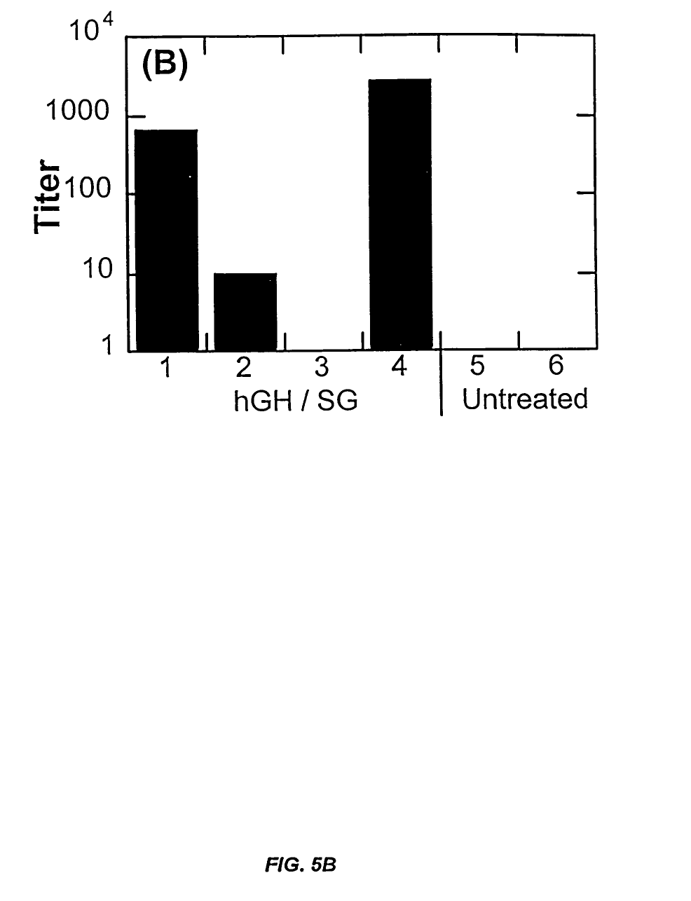

There is often no direct correlation between plasma IgA and mucosal secretory IgA (sIgA) (see, e.g., Russell et al., Infect. Immun. 64:1272 (1996)). To determine whether elevated plasma IgA titers could parallel a broad mucosal immune response within the RSGV animals or whether, the IgA response may have been strictly monomeric, and not secreted at mucosal sites, the saliva of RSGV and unvaccinated animals was examined for the presence of hGH specific IgA. Rats were given a single retroductal administration of 350 μg hGH DNA and saliva was collected after 3 weeks. Specific anti-hGH IgA responses were significantly elevated in the saliva of RSGV animals compared to untreated animals (FIG. 5A). To exclude the possibility that monomeric IgA in the blood is simply leaking through the gland, as opposed to being actively transported, the amount of specific antibody containing the secretory component was quantified; dimeric IgA binds to the poly Ig receptor and then is transported to the luminal side for release into the saliva (see, e.g., Brandzaeg et al., Gut 29:1116 (1988)). The measured anti-hGH secretory titers paralleled the results of the anti-hGH IgA (FIG. 5B). These results suggested that substantial amounts of hGH specific sIgA were being generated in the vaccinated animals.

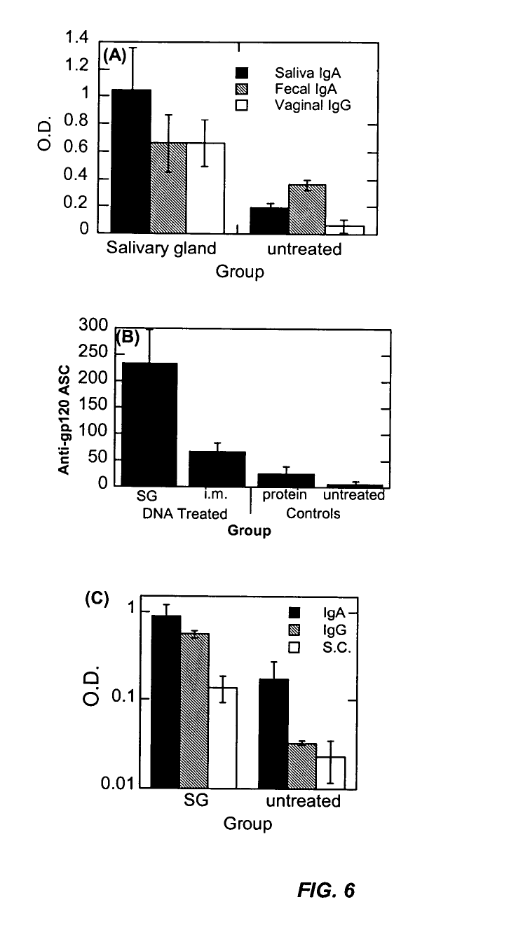

It has been reported that retroductal infusion of soluble protein antigens into the SG of monkeys induces detectable levels of specific antibodies of IgA isotype in saliva (see, e.g., Emmings et al., Infect. Immun. 12(2): 281-292 (1975)). To determine if DNA vaccination could also induce a mucosal immune response, specific IgA within the saliva of RSGV and unvaccinated animals was examined. Rats were given gp120 DNA 0 and 3 weeks, and saliva was collected directly from the submandibular gland through a cannula following pilocarpine injection. Pilot studies suggested that DNA co-formulated with Lipid/Zn may be more effective at eliciting specific saliva IgA, so this formulation was used. Results demonstrated a significant IgA response within the saliva of RSGV vaccinated rats (FIG. 6A).

To determine whether the mucosal response extended to distal mucosal tissues (e.g., intestines, lungs, and vagina), anti-gp120 IgA was first measured from fecal samples at week 6, using the same animals described above for the saliva analysis. The average O.D. of the fecal samples for the RSGV rats was greater than the O.D. of untreated animals (FIG. 6A). Additional methods were also used to characterize the intestinal response to antigen. Peyer's patches are part of the gut associated lymphoid tissue (GALT), and IgA secreted from the Peyer's patches contributes significantly to the total IgA found within the lumen of the small intestine (see, e.g., Brandzaeg et al., 1988, supra)). Animals were treated with DNA encoding gp120 by RSGV or by i.m. injection on weeks 0, 4, and 8 (same animals as described for the γ-IFN intracellular T cell assay in FIG. 4B). Cells were isolated from the Peyer's patches and the numbers of IgA producing cells from the Peyer's patches were measured one week after the last DNA delivery (see, e.g., Williams et al., J. Immunol. 161(8): 4227-4235 (1998)). Evaluation of cells isolated from Peyer's patches on week 9 suggested that RSGV induced a substantial IgA response (FIG. 6B). The mean number of anti-gp120 IgA antibody secreting cells (ASC) was 233 per 1.5×10⁶ Peyer's patch cells following salivary gland genetic vaccination, significantly more than the ASC detected in animals that received either i.m. DNA or s.c. protein injections. The results from the protein inoculation were particularly interesting since these animals had similar plasma IgA responses to RSGV, yet protein vaccination produced few ASC in the Peyer's patches (FIG. 6B). These results support the possibility that there is not necessarily a correlation between plasma IgA and mucosal IgA. Salivary gland vaccination promoted both types of IgA responses whereas protein vaccination stimulated only plasma IgA.

The immune response was measured within the vaginal and lung washes at 19 and 21 weeks respectively. Animals were vaccinated by RSGV on weeks 0 and 3 using the Zn/Lipid based formulation described before. Besides IgA, specific IgG plays a significant role in vaginal and lung mucosal immunity (see, e.g., Russell, Am. J. Reprod. Immunol. 47(5): 265-268 (2002)). Specific IgA and IgG responses to gp120 were measured in lung lavages (FIG. 6C). Antibodies that recognize gp120 were present in the lung lavages of RSGV animals, with significantly higher O.D. values for IgA isotype responses over untreated animals. Dimeric IgA at mucosal surfaces contains the secretory component, a remnant of active transport of the antibody through epithelial cells of the mucosa, whereas the majority of IgA in blood is monomeric in form and lacks the secretory component (see, e.g., Brandtzaeg, J. Reprod. Immunol. 36(1-2): 23-50 (1997)). The secretory component results parallel the IgA responses; animals with high specific IgA O.D. values also had high specific secretory component O.D. values (FIG. 6C). In contrast to the lungs, vaginal washes in our experimental animals contained almost no IgA and significant amounts of total IgG. The vaginal washes contained less than 4 ng/ml IgA whereas the amount of total IgG was above 350 ng/ml. For this reason, only specific IgG was measured in the vaginal washes. Anti-gp120 IgG values were found to be significantly above the values from the naive animals (FIG. 6A). These results demonstrate that RSGV vaccination can induce significant mucosal antibody responses distally from the site of DNA administration.

### Example 5

### Induction of Anti-hGH IgG in Dogs

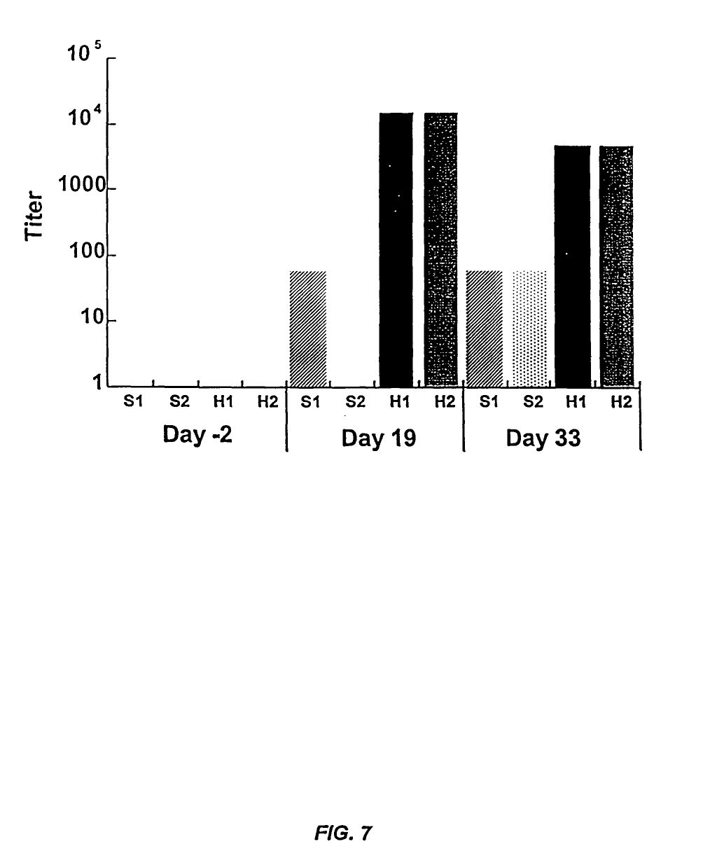

On day 0, 2.5 mg of plasmid DNA encoding hGH or 2.5 mg of plasmid DNA encoding secreted alkaline phosphatase (SEAP) was retroductally delivered to the parotid salivary glands of 10 kg dogs in a total volume of 700 μl with 2 mg/ml Evans Blue. On day 7, 0, 5.25 mg of plasmid DNA encoding hGH or 5.25 mg of plasmid DNA encoding secreted alkaline phosphatase (SEAP) was retroductally delivered to the parotid salivary glands of 10 kg dogs in a total volume of 3000 μl with 2 mg/ml Evans Blue. Anti-hGH IgG was measured 2, 19, and 33 days after the second infusion of DNA. N=2 for the hGH DNA group, N=2 for the unrelated antigen group. Antibody titers to hGH protein were greater than 5,000. The results are shown in FIG. 7. These results support the proposition that RSGV will scale up in direct proportion to body size.

### Example 6

### Coformulation of DNA with Lipids Enhances the Potency of Genetic Immunization

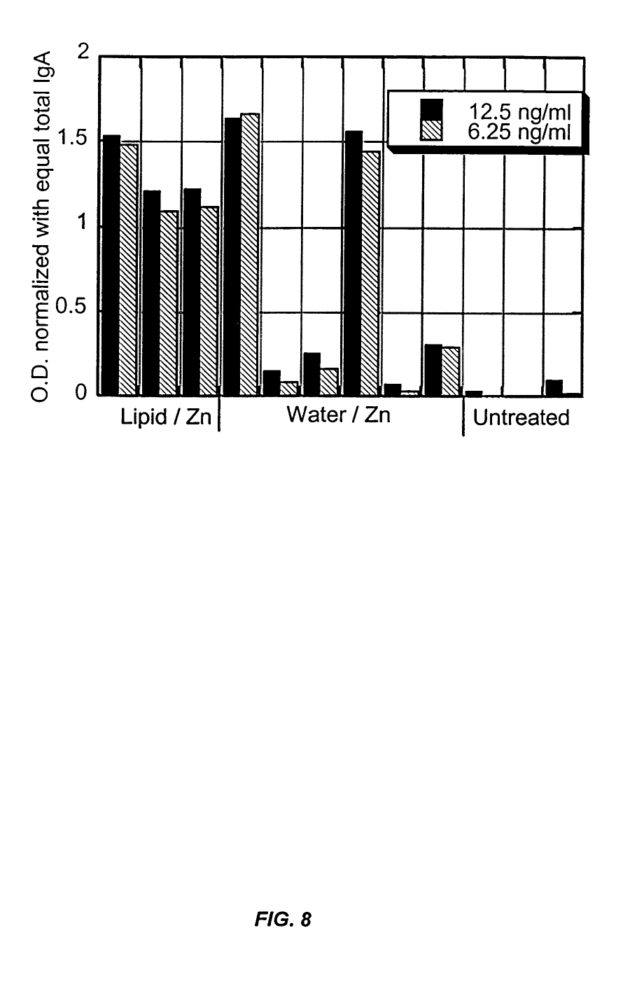

DNA encoding hGH was co-formulated with ZnCl₂ alone or with ZnCl₂ and 200 μg DOHBD:DOPE (3:1), then retroductally delivered to submandibular salivary glands of Sprague-Dawley rats at weeks 0 and 6. 88 μg DNA encoding hGH was administered per submandibular salivary gland. Nine weeks after delivery, hGH specific IgA titers were analyzed. The results are shown in FIG. 8.

### Example 7

### Dendritic Cells are Transfected by Retroductal Introduction of Compositions Comprising Nucleic Acids into the Lumen of the Salivary Gland Duct

175 μg DNA encoding green fluorescent protein or HIV envelope protein (i.e., gp120) and Evan's blue (4 mg/ml) in a total volume of 200 μl , was retroductally introduced into the lumen of the submandibular salivary gland of Sprague Dawley rats. Twenty four hours after introduction of DNA, cells were isolated from the draining lymph nodes and stained with antibodies specific for the dendritic cell specific markers CD86, CD11, as well as gp120. Flow cytometry analysis was used to measure expression of gp120 and GFP in the stained cells. The results show that dendritic cells in the draining lymph nodes are transfected by nucleic acids encoding GFP and gp120 when the nucleic acids are retroductally administered according to the methods of the present invention.

### Example 8

### Anti-Anthrax Protective Antigen Response Following Genetic Immunization

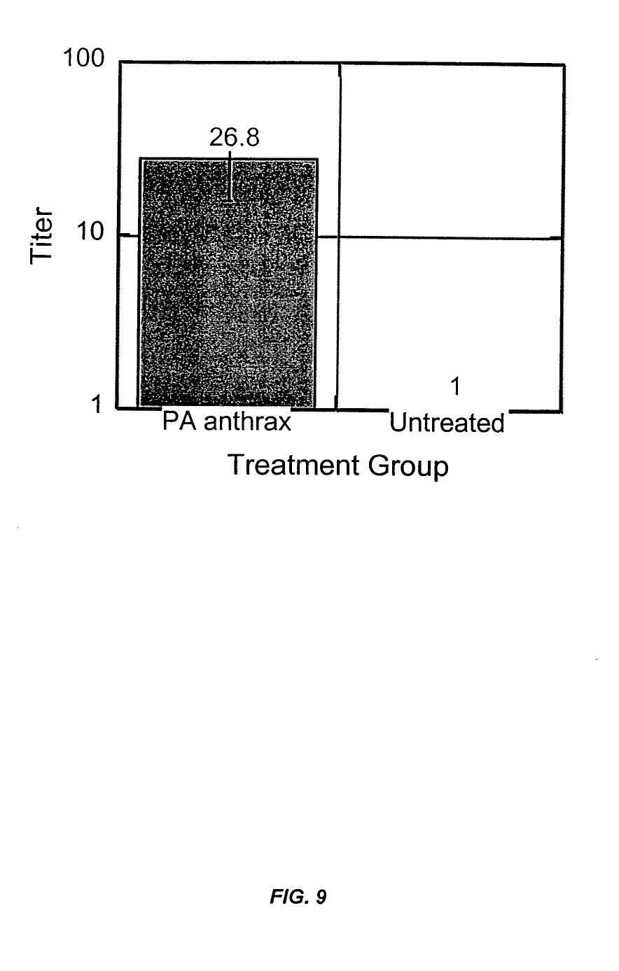

175 μg DNA encoding anthrax protective antigen (PA) in 200 μl water with 4 mg/ml Congo Red was retroductally introduced into the lumen of the submandibular salivary gland duct of Sprague/Dawley rats on week 0. The PA specific antibody titers were examined 3 weeks later to detect plasma antibody responses. N=6 for DNA vaccinated, N=3 for untreated. Anti-PA (anthrax protective antigen) response is seen following retroductal DNA delivery. The results are shown in FIG. 9.

### Example 9

### Distal Mucosal Immune Response Following Genetic Immunization

100 DNA encoding hGH (i.e., plasmid pFOXCMVhuGH-G3) in 100 μl distilled, deionized H20 was retroductally delivered to the submandibular salivary glands of Sprague Dawley rats on weeks 0 and 8. On week 12, lung lavages were collected. Briefly, the thoracic cavity was exposed and the vena cava and aorta severed to drain blood. A small incision was made on the major bronchi and 1 ml of PBS was injected into the lung via the bronchi, and then the fluid was withdrawn. This was repeated 3 to 5 times, and the fluid collected on ice and then diluted at 1/10 volume of sample volume immediately. After centrifugation at 4000×rpm for two minutes the supernatants were collected and stored at −80° C.

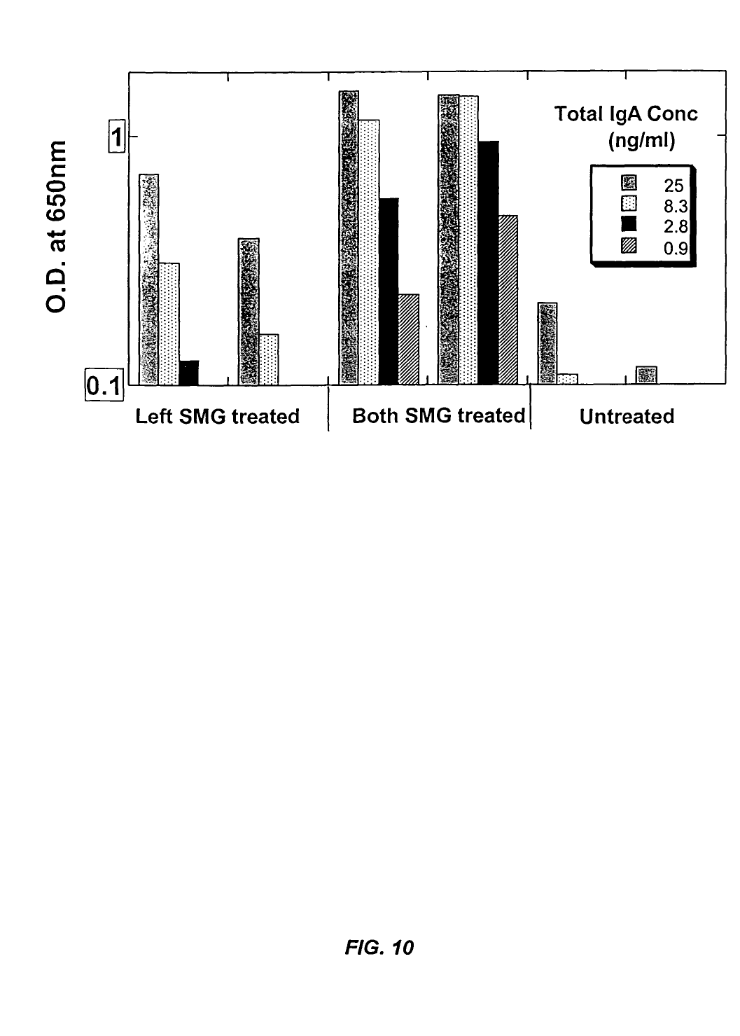

Anti-hGH IgA was detected using an ELISA as follows. Briefly, 96 well microtiter plates were coated with hGH at 1 ug/ml. 25 ng/mL of lung lavage supernatant was added to each well of the first row of hGH coated ELISA plate. Subsequent rows received serially diluted samples, diluted 1:3 from 25 ng/mL to 0.01 ng/mL with PBST. Sample dilution was based on the concentration of total IgA content of each lung lavage sample as determined by non-specific rat IgA ELISA. The secondary is goat anti-Rat IgG HRP at 1:2000 dilution. The results are shown in FIG. 10.

Taken together, the data presented here suggest that DNA immunization via retroductal delivery to the salivary glands is a potentially useful vaccine platform. Immune responses after gene transfer to the salivary glands have been reported. For example, Adesanya et al. have reported inflammation of the salivary glands after retroductal delivery of adenoviral vectors to rats (see, e.g., Adesanya et al., Hum. Gene Ther. 7:1085 (1996)). Kawabata et al. report that needle injection of DNA in mice, through the submucosal tissue and into the salivary gland, can produce humoral responses against the encoded protein (see, e.g., Kawabata et al., Infect. Immun. 67:5863 (1999)). The needle injection technique described by Kawabata et al. is similar to i.m. or s.c. in that the DNA is likely poorly distributed within the target tissue. The more robust immune responses (e.g., T cell activity and distal mucosal responses) detected after retroductal delivery of DNA to the lumen of the salivary gland duct according to the methods of the present invention, may be derived from the perfusion of the gland with DNA solution. The results presented here reveal that needle injection into the submucosa yields immune activity that is inferior to retroductal perfusion of the gland (FIG. 2B).

### Example 10

### HIV Neutralization Following Genetic Immunization

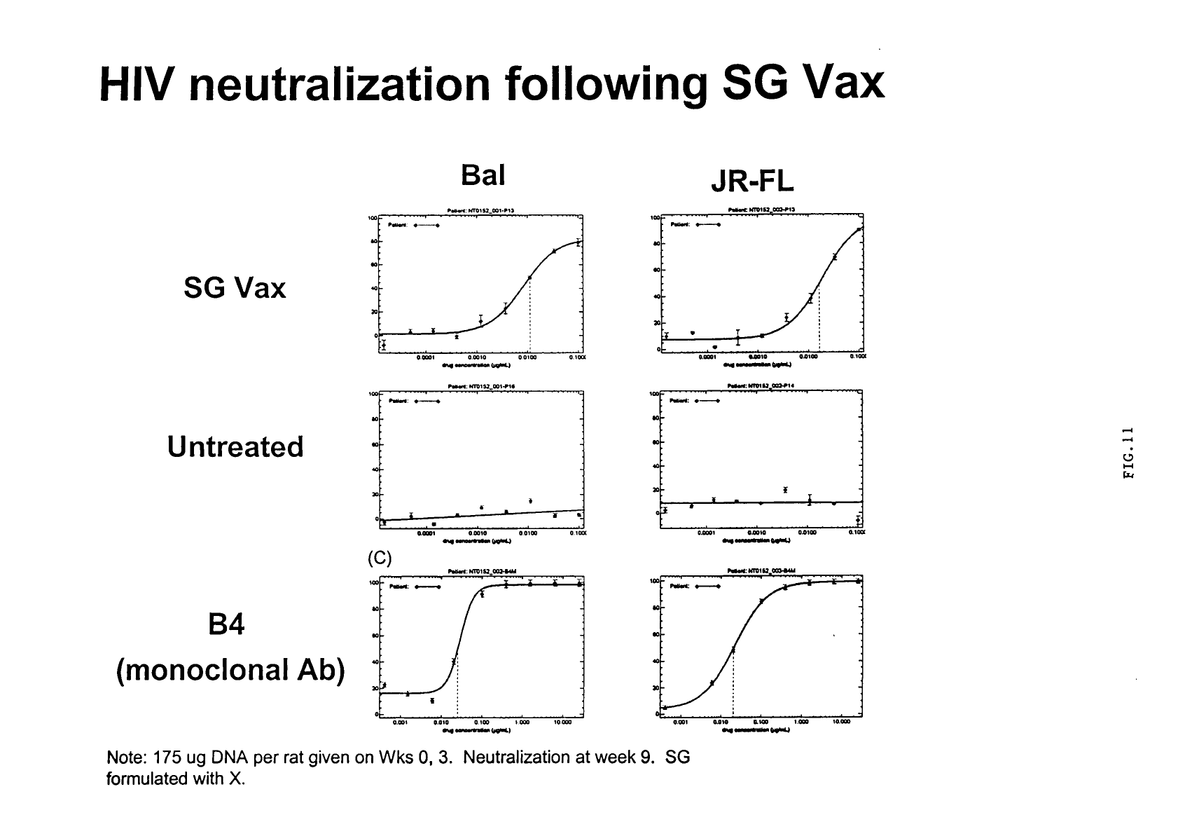

88 μg DNA encoding HIV envelope protein gp120 in 200 μl distilled, deionized H₂0 was retroductally delivered (50 μl/min.) to the submandibular salivary glands of Sprague Dawley rats on weeks 0 and 3. The DNA was delivered alone or in a formulation comprising: Congo Red (6 mg/ml), Congo Red (6 mg/ml )/DOHBD:DOPE (3:1)/Zn (0.125 mM), or aurintricarboxylic acid/Zn (0.125 mM). Anti-gp120 IgG titer was measured by ELISA over 17 weeks. All formulations were able to generate significant antibody responses to gp120 protein. On week 9, plasma samples were collected and HIV neutralization assays were performed using HIV strains Bal, JR-FL, MN, and IIIB. FIG. 11 depicts data demonstrating neutralization of Ba1 and JR-FL.

### Example 11

### Genetic Vaccination Protects Against Lethal Anthrax Challenge

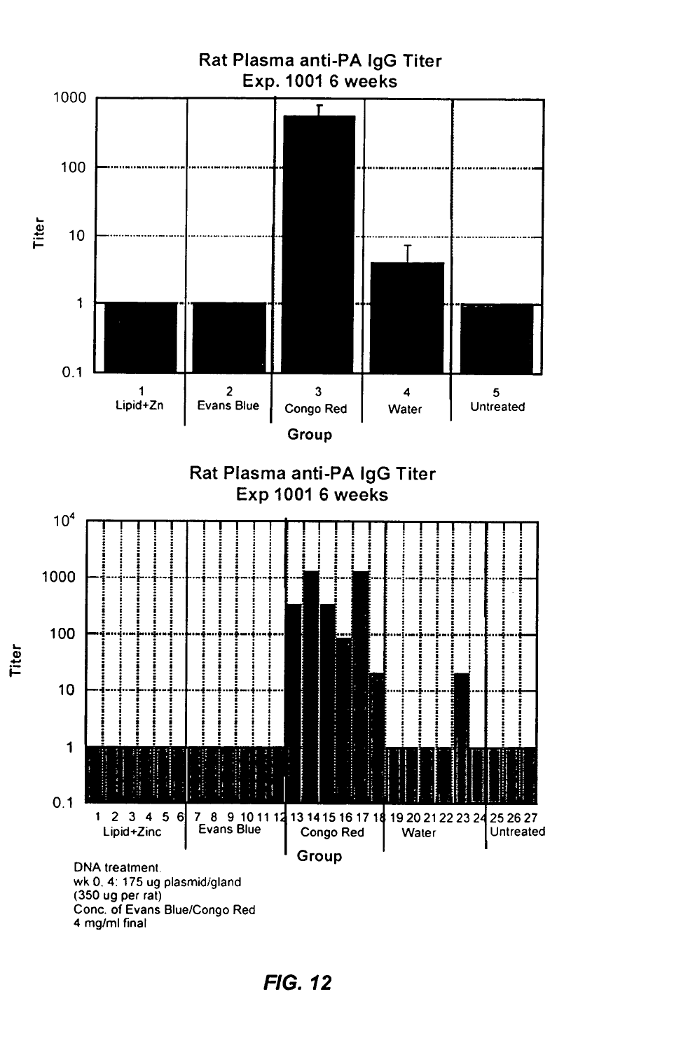

88 μg DNA encoding anthrax protective antigen (PA) in 200 μl with 4 mg/ml Congo Red was (1) retroductally introduced (50 μl/min.) into the lumen of each submandibular salivary gland duct or (2) intramuscularly introduced into the leg of Sprague/Dawley rats on week 0, 3, 6, and 9. DNA was formulated in compositions comprising water; Zn (0.125 mM)/DOHBD:DOPE lipid (3:1); Evans Blue (4 mg/ml); or Congo Red (4 mg/ml). Anti-PA IgG titer was measured by ELISA over 14 weeks. As shown in FIG. 12, DNA formulated in compositions comprising Congo Red induced antibody titers above 1000. The Congo Red-treated animals were then challenged with 10× the minimum lethal dose of anthrax toxin. Table 1 shows that all animals with antibody titers above 5000 survived while the negative control animals all died within 2 hours.

TABLE 1
Anthrax Challenge Experiment No. 1

| | Site of | | Survival 2 | Survival 4 | Survival 24 |
|---|---|---|---|---|---|
| Animal | vaccine | IgG Titer | hours | hours | hours |
| 1 | Salivary | 20480 | Alive | Alive | Alive |
| | gland | | | | |
| 2 | Salivary | 5120 | Alive | Alive | Alive |
| | gland | | | | |
| 3 | Salivary | 5120 | Alive | Alive | Alive |
| | gland | | | | |
| 4 | Salivary | 1280 | Alive | Alive | Alive |
| | gland | | | | |
| 5 | Salivary | 1280 | Alive | Dead | Dead |
| | gland | | | | |
| 6 | Salivary | 1280 | Alive | Dead | Dead |
| | gland | | | | |
| 7 | Untreated | 1 | Dead | Dead | Dead |
| 8 | Untreated | 1 | Dead | Dead | Dead |
| 9 | Untreated | 1 | Dead | Dead | Dead |

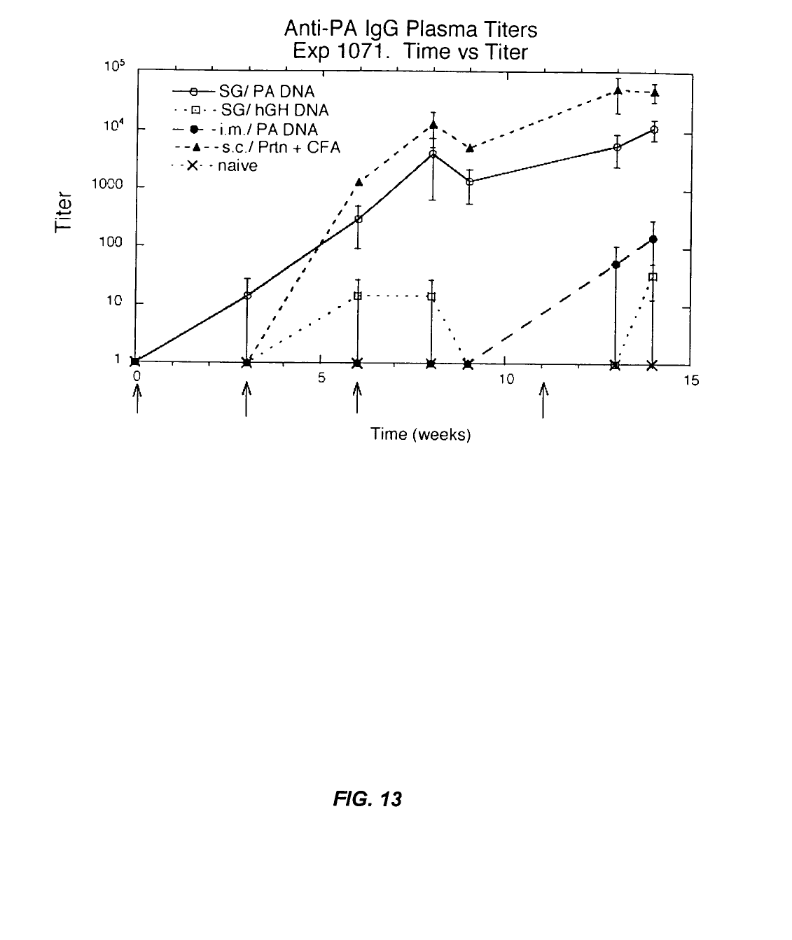

In a similar experiment, animals were treated by (1) retroductally introducing DNA encoding anthrax protective antigen (PA) or human growth hormone (hGH) into the salivary gland (SG), (2) intramuscularly (i.m.) introducing DNA encoding PA, or (3) subcutaneously (s.c) injecting PA protein with complete Freund's adjuvant (CFA). Antibody responses to PA were mesured over time following initial vaccination. As shown in FIG. 13, the highest anti-PA IgG plasma titers were observed for SG introduction of DNA encoding PA or s.c. injecting of PA protein. Arrows indicate when either DNA or protein was administered to the animals.

On week 14 (5 weeks after the last vaccination), mice were challenged with 10× the minimum lethal dose of anthrax toxin. All intramuscularly vaccinated, negative control, and irrelevay DNA (hGH) vaccinated animals died within 3 hours (Table 2). In contrast, 4 out of 5 SG vaccinated animals with titers above 1000 survived for longer than 24 hours and had no clinical signs of exposure to the toxin. The results are shown in Table 2 below:

TABLE 2
Anthrax Challenge Experiment No. 2

| | | | Survival | Survival | Survival |
|---|---|---|---|---|---|
| | Site of vaccine | IgG | 2 | 4 | 24 |
| Animal | Antigen | Titer | hours | hours | hours |
| 1 | Salivary gland | 24300 | Alive | Alive | Alive |
| | Anthrax PA DNA | | | | |
| 2 | Salivary gland | 900 | Alive | Dead | Dead |
| | Anthrax PA DNA | | | | |
| 3 | Salivary gland | 8100 | Alive | Alive | Alive |
| | Anthrax PA DNA | | | | |
| 4 | Salivary gland | 24300 | Alive | Alive | Alive |
| | Anthrax PA DNA | | | | |
| 5 | Salivary gland | 8100 | Alive | Alive | Dead |
| | Anthrax PA DNA | | | | |
| 6 | Salivary gland | 2700 | Alive | Alive | Alive |
| | Anthrax PA DNA | | | | |
| 7 | Salivary gland | 100 | Alive | Dead | Dead |
| | hGH DNA | | | | |
| 8 | Salivary gland | 100 | Dead | Dead | Dead |
| | hGH DNA | | | | |
| 9 | Salivary gland | 1 | Dead | Dead | Dead |
| | hGH DNA | | | | |
| 10 | Salivary gland | 1 | Dead | Dead | Dead |
| | hGH DNA | | | | |
| 11 | Salivary gland | 1 | Dead | Dead | Dead |
| | hGH DNA | | | | |
| 12 | Salivary gland | 1 | Dead | Dead | Dead |
| | hGH DNA | | | | |
| 13 | Intramuscular | 1 | Alive | Dead | Dead |
| | Anthrax PA DNA | | | | |
| 14 | Intramuscular | 1 | Dead | Dead | Dead |
| | Anthrax PA DNA | | | | |
| 15 | Intramuscular | 800 | Alive | Dead | Dead |
| | Anthrax PA DNA | | | | |
| 16 | Intramuscular | 1 | Dead | Dead | Dead |
| | Anthrax PA DNA | | | | |
| 17 | Intramuscular | 1 | Dead | Dead | Dead |
| | Anthrax PA DNA | | | | |
| 18 | Intramuscular | 1 | Dead | Dead | Dead |
| | Anthrax PA DNA | | | | |
| 19 | Untreated | 1 | Dead | Dead | Dead |
| 20 | Untreated | 1 | Dead | Dead | Dead |
| 21 | Untreated | 1 | Dead | Dead | Dead |
| 22 | Subcutaneous | 24300 | Alive | Alive | Alive |
| | Anthrax PA protein | | | | |
| 23 | Subcutaneous | 72900 | Alive | Alive | Alive |
| | Anthrax PA protein | | | | |

### Example 12

### hGH Expression in Tissue and Anti-hGH Responses in the Plasma of Rats Following RSGV

Material and Methods: The experiments were performed as described in Example 1 above with the following modifications and additions. There were 6 animals per DNA vaccinated group, 3 per protein vaccinated group, and 3 per untreated group unless otherwise stated. Lung lavages were collected after first perfusing and draining the circulatory system with PBS and then washing the interior of the lungs with 1 ml PBS. By removing blood before lung lavage collection, any contamination of the lung washes with plasma antibodies was avoided.

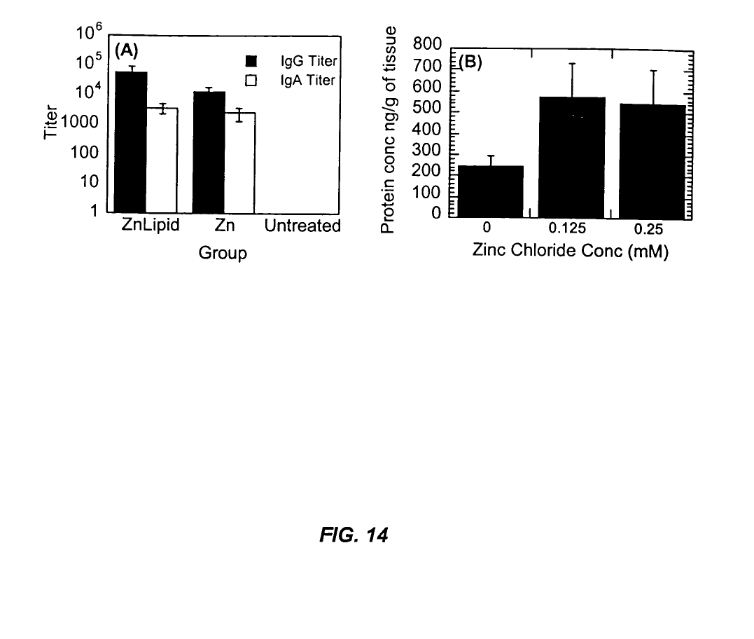

Antibody Responses By Salivary Gland Vaccination: Titers above 10,000 are induced in the plasma following retroductal administration of plasmid DNA encoding an antigen formulated either with H₂O, Zn, or a cationic liposome formulation with Zn (Znlipid) to rat submandibular glands (SMG) (FIG. 14A). Addition of Zn improved protein expression compared to the lipid based formulation (FIG. 14B), but did not improve systemic antibody titers significantly (FIG. 14A). These results demonstrate that high-titer and functional antibody responses are generated following SG DNA vaccination.

DNA Distribution And Scalability Following Retroductal Delivery Of DNA To The Salivary Glands: Intramuscular DNA vaccination is limited by the distribution of the inocula, and expression of the transgene post administration (see, e.g., Dupuis et al., J. Immunol. 165(5): 2850 (2000)). These effects are magnified in large animals, which may account for the poor performance in human studies. Because of the ductal structure of the salivary glands, retroductal delivery of DNA to the salivary glands provides an attractive alternative. We have shown that retroductal gene transfer promotes protein expression within the glands and that more than 99.9% of the DNA was contained with the capsular structure of the glands with less than 0.1% of the DNA reaching the draining lymph nodes as determined by q-PCR. Because of the favorable DNA distribution profile and because the ratio between the size of the animal to the size of salivary gland is roughly proportional, the ability to effectively vaccinate large animals should not be hindered by poor gene transfer.

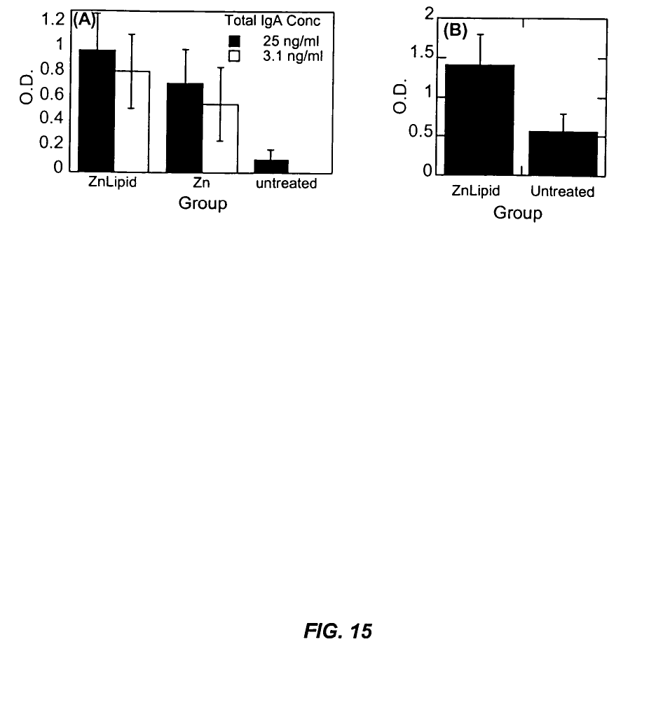

Mucosal Immune Responses In Saliva, Lungs, And Intestinal Mucosa: After vaccination by retroductal gene transfer to the SG, the immune response was assessed at a variety of mucosal tissues. Salivary IgA responses were compared using either the Zn or ZnLipid formulation. Results were statistically indistinguishable between the 2 formulations at two different concentrations of total IgA, but both formulations were well above background (FIG. 15A). Lung IgA reponses were measured using the ZnLipid formulation in a separate experiment and show a similar stimulatory response (FIG. 15B). Thus, SG DNA vaccination induces detectable mucosal IgA both locally and distally.

### Example 13

### Enhancement of Mucosal Immune Responses to HIV Envelope Protein following DNA vaccination to the SMG

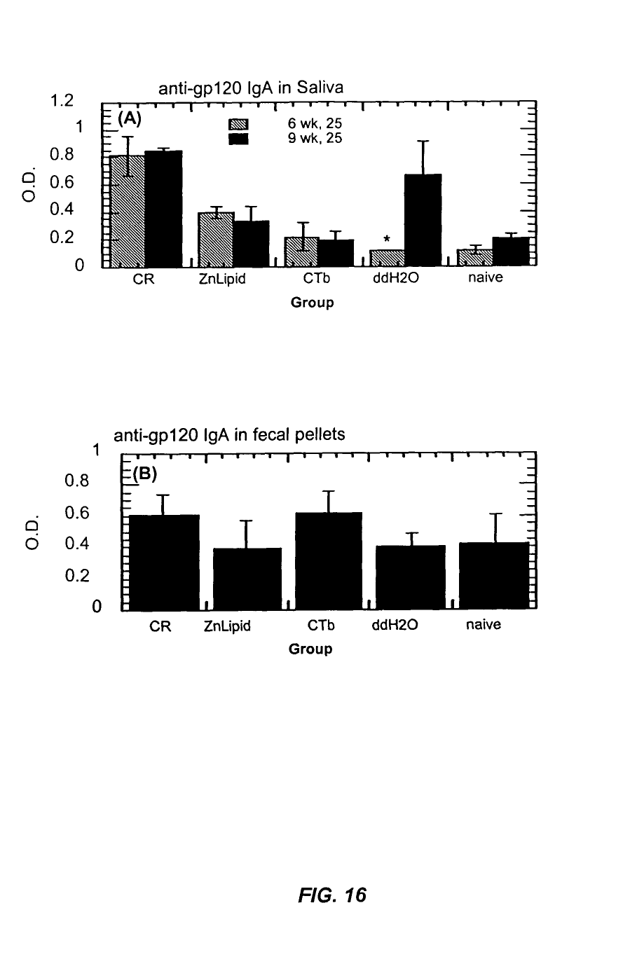

Cholera toxin B subunit (CTb) to a Zn Lipid formulation (ZnLipid), water, and Congo Red (CR) for the ability to improve systemic and mucosal immune responses. These responses were measured by examining plasma, saliva and fecal pellets for specific antibodies. ZnLipid and Congo Red appear to be more effective than CTb for eliciting both systemic and salivary IgA responses (FIG. 16A). However, the fecal results appear to show potentially equivalent results between CTb and CR formulations. Many of these animals had elevated O.D. values (FIG. 16B).

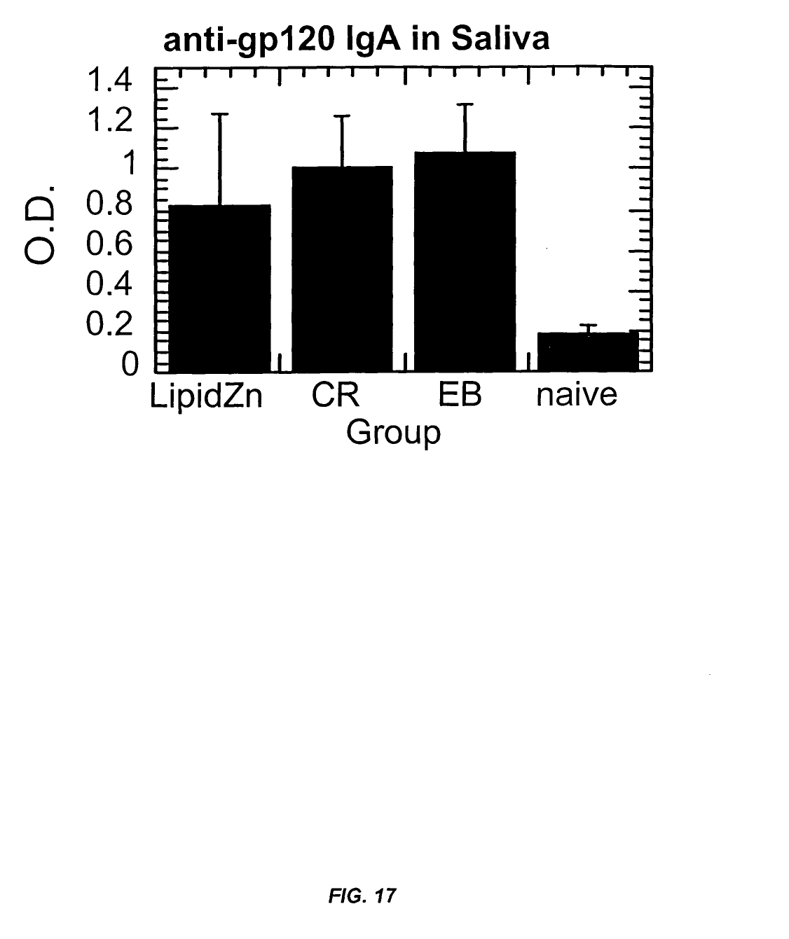

In an additional experiment, many of our expression enhancing adjuvants were evaluated. Both CR and Evans blue (EB) appear to perform equivalently in inducing saliva IgA responses (FIG. 17) and in systemic antibody titers to gp120.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed is:

1. A method for eliciting an immune response, the method comprising retroductally introducing into the lumen of a salivary gland duct of a subject an immunogenically effective amount of a composition comprising a nucleic acid encoding an immunogenic polypeptide, whereby an immune response is generated.

2. The method of claim 1, wherein the step of delivering is by cannulation.

3. The method of claim 1, wherein the composition further comprises an adjuvant.

4. The method of claim 3, wherein the adjuvant is a cholera toxin.

5. The method of claim 3, wherein the adjuvant is Al(OH)₃.

6. The method of claim 3, wherein the adjuvant is a lipid.

7. The method of claim 3, wherein the adjuvant is a polyionic organic acid.

8. The method of claim 7, wherein the polyionic organic acid is 6,6′-[3,3′-demithyl[1,1′-biphenyl]-4,4′-diyl)bis(azo)bis[4-amino-5-hydroxy-1,3-naphthalene-disulfonic acid].

9. The method of claim 1, wherein the composition is administered multiple times.

10. The method of claim 1, wherein the nucleic acid is operably linked to an expression control sequence.

11. The method of claim 1, wherein the nucleic acid is in a viral vector.

12. The method of claim 1, wherein the immunogenic polypeptide is a cancer antigen.

13. The method of claim 1, wherein the immunogenic polypeptide is a viral antigen.

14. The method of claim 13, wherein the viral antigen is HIV envelope protein or a portion thereof.

15. The method of claim 1, wherein the immunogenic polypeptide is a bacterial antigen.

16. The method of claim 15, wherein the bacterial antigen is anthrax protective antigen.

17. The method of claim 3, wherein the composition further comprises a lipid, whereby the lipid facilitates uptake of the nucleic acid by antigen presenting cells.

18. The method of claim 17, wherein the lipid is N,N,N′,N′-tetramethyl-N,N′-bis(2-hydroxyethyl)-2-3-di(oleoyloxy)-1,4-butanediammonium iodide.

19. The method of claim 1, wherein the salivary gland is a submandibular salivary gland.

20. The method of claim 1, wherein the salivary gland is a parotid salivary gland.

21. The method of claim 1, wherein the salivary gland is a sublingual salivary gland.

22. The method of claim 1, wherein the subject is a mammal.

23. The method of claim 22, wherein the mammal is a primate.

24. The method of claim 23, wherein the primate is a human.

25. The method of claim 1, wherein the immune response comprises a mucosal immune response.

26. A method for transfecting antigen presenting cells, the method comprising retroductally introducing into the lumen of a salivary gland duct of a subject an immunogenically effective amount of a composition comprising a nucleic acid encoding an immunogenic polypeptide, whereby an antigen presenting cell is transfected with the nucleic acid.

27. The method of claim 26, wherein the step of delivering is by cannulation.

28. The method of claim 26, wherein the composition is administered multiple times.

29. The method of claim 26, wherein the nucleic acid is operably linked to an expression control sequence.

30. The method of claim 29, wherein the nucleic acid is in a viral vector.

31. The method of claim 26, wherein the immunogenic polypeptide is a cancer antigen.

32. The method of claim 26, wherein the immunogenic polypeptide is a viral antigen.

33. The method of claim 32, wherein the viral antigen is HIV envelope protein or a portion thereof.

34. The method of claim 26, wherein the immunogenic polypeptide is a bacterial antigen.

35. The method of claim 34, wherein the bacterial antigen is anthrax protective antigen.

36. The method of claim 26, wherein the composition further comprises a lipid, whereby the lipid facilitates uptake of the nucleic acid by the antigen presenting cells.

37. The method of claim 26, wherein the salivary gland is a submandibular salivary gland.

38. The method of claim 26, wherein the salivary gland is a parotid salivary gland.

39. The method of claim 1, wherein the salivary gland is a sublingual salivary gland.

40. The method of claim 26, wherein the subject is a mammal.

41. The method of claim 40, wherein the mammal is a primate.

42. The method of claim 41, wherein the primate is a human.

43. The method of claim 26, wherein the antigen presenting cells in a proximal lymph node are transformed by the nucleic acid.

44. The method of claim 43, where in the antigen presenting cells are dendritic cells.

45. The method of claim 43, wherein the proximal lymph node is a draining lymph node.

46. The method of claim 43, wherein the draining lymph node is a submandibular lymph node.

47. The method of claim 43, wherein the draining lymph node is a parotid lymph node.

48. The method of claim 43, wherein the draining lymph node is a cervical lymph node.

49. The method of claim 26, wherein the antigen presenting cells in a salivary gland are transformed by the nucleic acid.

50. A pharmaceutical composition, the composition comprising:
a nucleic acid encoding an immunogenic polypeptide;
a lipid; and
a transition metal enhancer.

51. The composition of claim 50, wherein the lipid is N,N,N′,N′-tetramethyl-N,N′-bis(2-hydroxyethyl)-2-3-di(oleoyloxy)-1,4-butanediammonium iodide and the transition metal enhancer is ZnCl₂.
